(19) United States
(12) Patent Application Publication
(10) Pub. No.: US 2003/0207307 A1
(43) Pub. Date: Nov. 6, 2003

(54) Determining biochemical markers of progression and therapy monitoring and specification, therapeutic lead molecules, and target biochemical systems applied to stroke

(75) Inventor: Wayne R. Matson, Ayer, MA (US)

Correspondence Address: Norman P. Soloway, HAYES SOLOWAY P.C., 130 W. Cushing Street, Tucson, AZ, 85701, US

(73) Assignee: ESA, Inc.

(21) Appl. No.: 10/410,996

(22) Filed: Apr. 10, 2003

Related U.S. Application Data
(63) Continuation-in-part of application No. 10/366,909, filed on Feb. 14, 2003.
Application No. 10/366,909 is a division of application No. 09/687,047, filed on Oct. 13, 2000, now Pat. No. 6,548,252.
(60) Provisional application No. 60/373,449, filed on Apr. 18, 2002.

(51) Int. Cl. C12Q 1/68; C07H 21/04

(52) U.S. Cl. 435/6; 536/24.3

(57) ABSTRACT

Methods for determining the inventory of small process and control molecules (The Metabolome) and the interaction of the Metabolome with the Genome and Proteome (Metabolomic databases). Methods for evaluating Metabolomic databases for biochemical markers that correlate with outcome of a disease, allow monitoring of therapy and direct therapy. Methods for evaluating Metabolomic databases for lead therapeutic molecules and target systems for therapeutic manipulation. Methods of evaluating all relationships in a Metabolomic database for outcome of disease or therapy. The methods of Metabolomic database evaluation have application to stroke. Outcome predicting and therapy directing or monitoring markers of Methylation and oxidative processes in stroke have been determined. Methylation processes have been identified as targets for therapeutic development. A novel class of potential therapeutic agents that bind to DNA have been identified in stroke.

### CROSS-REFERENCE TO RELATED APPLICATIONS

This application is a continuation-in-part of my co-pending U.S. application Ser. No. 10/366,909, filed Feb. 14, 2003, which in turn is a divisional of U.S. application Ser. No. 09/687,047, filed Oct. 13, 2000, the disclosure of which is incorporated by reference. This application also claims benefit of U.S. Provisional Application Serial No. 60/373,449, filed Apr. 18, 2002.

### BACKGROUND OF THE INVENTION

In my aforesaid Parent application Ser. No. 09/687,047 (hereinafter my Parent Application), I describe an analytical technique for detecting cancer or other disease and/or health conditions based on measurement of free levels of nucleotide excision products resulting from DNA or RNA damage, such as 8OH2′dG—8 hydroxy 2′deoxyguanosine; O6MG—O-6-methylguanine; 2′dG—2′deoxyguanosine; 7MG—7-methylguanine; 8NG—8-nitroguanine; 8OHG—8-hydroxyguanine; 8OH2′dA—8-hydroxy-2-deoxyadenosine; 8OHA—8-hydroxyadenine; 5OH2′dCy—5-hydroxy 2′deoxycytidine; 5OHU—5-hydroxyuracil; 3NT—3-nitrotyrosine; or 3 CIT—3-chlorotyrosine in biological samples from selected areas of the body. More particularly, my invention described in my Parent Application is based on the hypothesis that specific areas of the body are semi-isolated in-situ biochemical environments for nucleotide excision products such as 8OH2′dG or other nucleotide excision products, and that the levels of such free 8OH2′dG or other nucleotide excision products in the semi-isolated environment magnify the combined defects of DNA or RNA damage and repair mechanisms. By way of specific example, the effective damage and repair rate increases in DNA in cancer, or pre-cancerous cells, has been found to be magnified by accumulation in the extra-cellular matrix in selected areas of the body of 8OH2′dG. For example, as applied to cervical cancer, the cervix has been found to be a semi-isolated in-situ biochemical environment which may be accessed through cervico vaginal lavage sampling. Thus, an objective analytical technique for determination of cervical cancer is provided.

More particularly, my Parent Application describes electrochemical analysis of 8 hydroxy 2′ deoxyguanosine (8OH2′dG) in cervico vaginal lavage or PAP smear swab samples. Analysis is accomplished using an electrochemical analytical system made in accordance with and following the general analytical procedures disclosed in PCT Application No. PCT/US98/22275, and as discussed in “A Carbon Column Based LCEC Approach to Routine 8-Hydroxy-2′-Doxyguanosine Measurements in Urine and Other Biological Matrices” (Bogdanov MB, et al. Free Rad. Ciol. Med. 27, 1227-1248, 1999).

The conventional hypothesis of hydroxyl radical DNA damage and repair and excretion of the hydroxylated adduct 8OH2′dG is that a hydroxyl radical reacts with the DNA causing hydroxylation of the deoxyguanosine at the 8 position. The damaged segment is then either excised by glycolysis as the 8OHG or by endonuclease excision as the 8OH2′dG5′monophosphate. The monophosphate is then dephosphorylated and the 8OH2′dG is rapidly excreted from the cell. The 8OH2′dG excreted to the extracellular matrix is then cleared rapidly from the body primarily via the kidney and excretion in the urine. 8OHG can also be produced as a result of attack on RNA and glycolysis.

According to my Parent Application, studies performed with the technologies described above have confirmed certain basic elements of this hypothesis as follows:

1. Dialysis studies of 8OH2′dG in extracellular matrix with probes placed in brain and muscle of rat and mouse to measure the rate of production and excretion compared with the free levels inside the cellular material confirm that excretion from the cells is rapid. Similarly, comparison of CSF, Plasma and urinary levels of ca 1:10:2000 in ca 100 ALS and control subjects confirm that the rate of clearance from the body is high. Studies of saliva or sweat vs. urine levels and studies showing highly elevated levels in plasma kidney dialysis patients confirm that urinary excretion is the primary mode of removal.

2. Studies of urinary levels of 8OH2′dG within individuals over diurnal weekly, monthly and yearly intervals indicate that the rate of DNA damage and repair is highly constant and characteristic of an individual. Similarly, studies of siblings and parents which show a significantly closer agreement of sibling values indicate that the rate of DNA damage is strongly determined by genetic factors.

Other studies, however, have indicated some basic difficulties with the simplistic model of damage and repair which have relevance to the use of DNA damage markers as diagnostic tools.

1. The simple model of increased production of hydroxyl radical leading to increased DNA damage is incomplete.

Comparison of total body rate of production of hydroxyl radical estimated by salicylate spin trappings showed no correlation in normal individuals, ALS patients and Freidrich's Ataxia FA patients of urine or plasma levels of 8OH2′dG. Nor was there any group elevation of total body hydroxyl radical production in FA or ALS although the 8OH2′dG levels were increased in CSF plasma or urine by 25-30%.

Urine levels of 8OH2′dG were significantly increased in individuals exposed to arsenic or o-toluidine or aniline although these materials play no direct role in the increase of hydroxyl radical production.

These studies suggest that conformational changes in DNA induced by exogenous or indogenous adducts or changes in sub cellular structures in proximity to DNA play a stronger role in increasing the rate of DNA damage than overall hydroxyl radial production. Also supporting this concept are studies I have performed showing no effect of simple antioxidants such as Ascorbate or Tocopherol on the levels of 8OH2′dG.

2. Relevant to the use of DNA damage rate measurements to cancer diagnosis I have observed that in C. Elegans culture there is a burst of 8OH2′dG production during the stage of rapid replication of the gonadal cells. In similar experiments I observed that a toxin (3 nitro propionic acid, 3NP) increased the rate of DNA damage during replication. When the toxin was removed after exposure the rate of damage was still maintained at a higher level indicating that the initiation of a higher rate of damage creates a biochemical state or feed back that persists beyond the time of the applied insult.

3. It has also been observed in human controls, ALS patients and FA patients and in C. Elegans culture that the levels of 8OH2′dG that remain in the DNA (measured by extracting and hydrolyzing the DNA) are only weekly correlated with the rate of output of the 8OH2′dG. This indicates that both damage and repair are up regulated simultaneously and thus that measures of the free levels of excised DNA damage product provide a much more robust indicator of disorder related DNA damage processes.

4. In situations where there is a high rate of damage and repair the conventional sequence of excision of the 8OH2′dG phosphate, dephosphorylation of the 8OH2′dG and excretion of the 8OH2′dG does not hold. In rapid cell replication in C. Elegans, in 3NP insulted C. Elegans and in Cervix cancer cells (as described below) the 8OH2′dG phosphates are directly excreted.

5. Sampling specific to the site of insult is of considerable importance. In studies of smoking related increases of urinary 8OH2′dG levels, I have found a statistically significant increase of only 11% in a cohort of ca 200 smokers vs. ca 300 control non-smokers. However, the free levels of 8OH2′dG in the extra-cellular matrix from pharyngeal swabs of smokers vs. non-smokers are elevated by a factor of 3x-4x when normalized against the cellular metabolite of tyrosine, 4-hydroxy phenyl lactic acid.

The synthesis of these studies and observations leading to the approach to cancer diagnosis is as follows:

1. When a cell begins extensive abnormal replication the changes in the conformation of the DNA during mitosis make it more susceptible to free radial damage and both increase the levels of excised damage products and change the nature of those products.

2. Sample sites which are semi-isolated in situ from physiological transport and excretion, reflecting the extracellular matrix around the affected cells will show highly increased levels of the repair products relative to the damage products remaining.

3. Levels of the repair products can be normalized against other cellular metabolites that are excreted to the extracellular matrix from metabolic processes that are not materially affected by free radical damage.

In my aforesaid Parent Application, I also discuss strong evidence for concurrent methylation increases in N methyl and NN dimethyl serotonin (NM5HT NNM5HT) and 7 methyl guanine (7MG) in plasma and CSF and reduction in the oxidative DNA damage marker 8-Hydroxy 2′Deoxyguanosine (8OH2′dG) in stroke and ischemia in humans. More particularly employing the analysis techniques of the present invention shows a reduction in CNS production of the oxidative DNA damage product 8-Hydroxy-2′-deoxyguanosine (8OH2′dG) and an increase in the production of methylated compounds in a cohort of stroke patients and a similar reduction of 8OH2′dG levels in animal stroke models. Thus, elucidation of the biochemical mechanisms of methylation and oxidative free radical damage to DNA and other cellular structures in stroke and related disorders, and the determination of the relationships of the biochemistry to stroke management, provides a rapid single assay technology for methylated, reactive oxygen and nitrogen species (ROS, RNS) markers in clinically accessible samples and samples relevant to mechanistic studies in animal models. This provides for clinical management of and the evaluation of pharmacological intervention in stroke.

Preliminary work with purine specific carbon column switching techniques as above described also demonstrates elevations of the DNA hydroxy radical damage marker 8-hydroxy-2′-deoxyguanosine in a number of neurodegenerative diseases (NDD). Progressive increases with disease have been shown in, e.g. amyotrophic laterial sclerosis. That is to say, preliminary work with carbon column switching LCEC technology in accordance with the present invention has shown that the ROS DNA damage marker 8-hydroxy-2′deoxyguanosine (8OH2′dG) is elevated in Parkinson's Disease (PD) (urine, plasma, and CSF), Alzheimer's Disease (AD) (plasma and urine, Huntington's Disease (HD) (urine), Freidrich's Ataxia (FA) (urine plasma) and that it is elevated and progressive with time in Amyotrophic Lateral Sclerosis (ALS) (urine plasma and CSF) but not in myopathies. Secondary evidence from the patterns of chromatograms determining 8OH2′dG indicate differences in unidentified purine adducts in urine, plasma and CSF among the neurodegenerative disorder (NDD) categories. While not wishing to be bound by theory, the overall hypothesis is as follows:

1. DNA damage products resulting from processes of methylation/ROS/RNS/RCLS are different among controls and NDD.

2. Ligand of endogenous/exogenous compounds with DNA play a role in increased DNA damage.

3. Excision and repair processes differ among controls and NDD.

4. The sites of attack of RS on DNA differ among controls and NDD.

As mentioned supra, other DNA and RNA damage markers and free radical markers may be assayed as indicators for the presence of or risk of various health disorders.

### SUMMARY OF THE INVENTION

This invention relates to analytical and mathematical methods for evaluating the small molecule inventory and interactions of process and control molecules that regulate the function of an organism. This small molecule inventory and its interactions is designated as the Metabolome. Overall the Metabolome comprises tens to hundreds of thousands of distinct chemical species including such compounds as transmitters and metabolites, toxins and inhibitors, indicators of protein and DNA repair and damage and indicators of energy generation processes. The Metabolome also interacts directly and indirectly with the expression of the genetic code of an individual (the Genome) and the proteins expressed by the Genome (the Proteome). Metabolomics is defined as a description of the interactions among and within the Metabolome, Genome and Proteome. Information on the levels of small molecules in the Metabolome and their interactions among themselves and with the Genome and the Proteome are defined as Metabolomic databases.

The conceptual vision in which this invention is described is that non-lethal but non-functional shifts in the Metabolomics of an individual are in fact a disease, and the first indication of the state of disease in that individual.

Thus, in this model diseases such as Alzheimer's, Parkinson's, Cancer, Type II diabetes etc. are a non-functional but non-lethal shift in the metabolomics of an individual. The clinical onset or symptoms of the disease-degeneration of specific brain areas, development of tumors etc. are the result of the shift in the metabolomics.

Shifts in an individuals metabolomics can be latent in the genetics of an individual and express over time, or be triggered by an environmental insult either acutely or over time. They can also, as in trauma or stroke, be caused by a significant physical failure or insult.

This invention relates to the application of specific techniques of metabolomic definition in stroke. The techniques include but are not inherently limited to use of electrochemical arrays for profiling of ca 1500 small molecules in plasma, urine and cerebrospinal fluid (CSF), the use of separation procedures for isolating undenatured DNA and evaluating the nature of the interaction of ca. 1000 small molecules of the metabolome with DNA, specific protocols for indication of DNA damage protection and repair, and techniques for evaluating the interaction of ca. 1000 small molecules with the proteomic complement of individual samples.

The techniques allow:

1. The definition of markers of metabolomic shifts that predict outcome.

2. The definition of metabolic pathways that are targets for therapeutic intervention in initial acute care.

3. The definition of markers that are useful in monitoring or selecting therapies in acute care.

4. The development of patterns of metabolomic shifts that predict sequellae of stroke.

5. The definition of target pathways for intervention to prevent long term sequellae in stroke.

6. A specific novel class of therapeutic agents that bind to and protect DNA in controls but not in stroke subjects.

7. A specific novel class of compounds that bind to and decrease the fidelity of expression of DNA in stroke but not in control subjects.

While not wishing to be bound by hypothesis, a conceptual biochemical model of stroke is useful as a guide to evaluation of markers, therapeutic targets and therapeutic leads.

In this model in the acute phase of stroke there is initially a massive oxidative free radical stress in the central nervous system. The usual protective measures are overwhelmed and unusual latent protective pathways are upregulated. One of these pathways is methylation. Methylation acts to protect the genomic complement by N methylating DNA. In stroke this process essentially becomes promiscuous and methylates other compounds in an abnormal fashion. Some of these such as methylated serotonin and indole derivatives are, in an oxidizing environment, capable of forming neurotoxic agents that inhibit recovery.

In the sequellae of stroke, biochemical feed back loops from changes to the expression of the genome establish a new set of relationships and signaling and process control that does not return to normal. The changes in this metabolomic profile of subjects post stroke is responsible for and correlated with subsequent sequellae such as increased risk of other cardiovascular events.

### BRIEF DESCRIPTION OF THE DRAWINGS

Further features and advantages of the present invention will be seen from the following description, taken in conjunction with the accompanying drawings, wherein:

FIGS. 1a-1b and 2a-2b are a series of graphs showing current over time of an electrochemical analysis of plasma and CSF, respectively, for stroke and control subjects;

FIGS. 3a-3b are graphs, similar to FIGS. 1a-1b and 2a-2b of segments of array pattern metabolomic of plasma from control vs. stroke subject showing methylated serotonin compounds;

FIG. 4 is a graph showing correlation of 8OH2′dG and methylation with DNA damage and repair; and

FIG. 5 is a graph showing level of methylation over time.

### DETAILED DESCRIPTION OF THE INVENTION

Sample sets:

Cohort one: 273 plasma samples from 26 subjects with stroke were acquired in pairs from the jugular vein and peripheral arterial blood at times ranging from onset to three weeks. Cohort one had initial assessments and MRI data at various times. The data generated by the various platforms described below on different sample fractions and preparations comprised approximately 4000 variables for consideration. Because of the strictures on significance imposed by the Bonferoni correction, data from Cohort one was used to generate hypotheses of differences in a limited number of apparently significant compounds and pathway relationships. These were subsequently tested in Cohort two. Additional sample sites and types and a more rigorous time schedule were also imposed.

Cohort two: 240 plasma samples and 50 CSF samples from 22 subjects with stroke were acquired under the following schedule: Day one, three, seven, fourteen and twenty one, left and right jugular vein, peripheral arterial and venous blood; Day one and three lumbar CSF. Cohort two also has day 1 MRI scans and progressive assessments on the NIH stroke scale at the time of acquisition of samples.

54 peripheral venous plasma samples were acquired from age matched controls. 40 control CSF samples were accessed from existing libraries.

Urine samples from a separate cohort of 23 patients were acquired.

The design of the sample acquisition allowed description not only of overall biochemical changes but also of changes occurring primarily in the CNS. In the first cohort where only one descending jugular vein was sampled general observations of overall CNS changes vs. peripheral changes could be made. In the second cohort with samples from both jugulars observations could be made of changes specific to the laterality of the stroke locus and changes in the periphery relative to arterial and venous blood. CSF changes in the second cohort could also be related on a temporal basis to the biochemical changes reflected in plasma.

Analytical and methodological protocols:

All samples were analyzed for total profiles under variations of electrochemical array conditions disclosed in my U.S. Pat. Nos. 4,863,873, 5,104,639, 6,194,217 and 6,210,970.

Specific analyses for markers of DNA damage protection and repair were carried out using technology and variants of methods disclosed in my Parent application Ser. No. 09/687,047 using carbon column switching systems as described in my PCT/US98/22275 using electrode preparation and sample concentration protocol as described in Bogdanov et al, supra, for 8OH2′dG, 8OHG, 7MG and O6MG both as free levels and as levels in DNA.

There were two novel adaptations of these technologies applied to the specific problem of high density acquisition of data.

1. The array was fitted with a peak suppressor gradient mixer device before the injector. This device consists of a column of ca. 0.5 in diameter by 1 inch long packed in the first 0.5 inch with ca 0.04-0.06 inch diameter porous carbon and co packed with 5 u C18 particles for the remainder of its length. This arrangement has the effect of trapping contaminants for the mobile phases of the electrochemical array system during a gradient and on release by the gradient spreading the contaminant signals over 3-4 min elution times making their signals transparent to the peak detection software. The device allows definition of useful signals in areas of a profile that would otherwise be obscured by the part per trillion contaminants that are inherent in any set of reagents.

2. It was recognized that the high resolving power of the carbon column switching system would allow a novel method of isolating DNA to determine levels of damage and protection. Rather than essentially denaturing the DNA by a series of harsh extractions we simply forced a disrupted sample through a 300,000 molecular weight cut off polysulfone membrane. The DNA, which will not pass this membrane was then subjected to extraction or a standard digestion with P1 endonuclease, or alkaline phosphatase, or HCl. It was further recognized that as well as being amenable to assay of specific DNA damage or protection indicators, such preparations were also amenable to total profiling.

In this study such separation and assay protocols were applied to both exfoliated cells in CSF and leukocytes to determine both the specific nature of DNA damage products and the overall profile of ca 1000 ligands bound to DNA. Comparison of extracted DNA, and digested DNA by various methods allows assessment of the nature and strength of various ligands.

Data assessment:

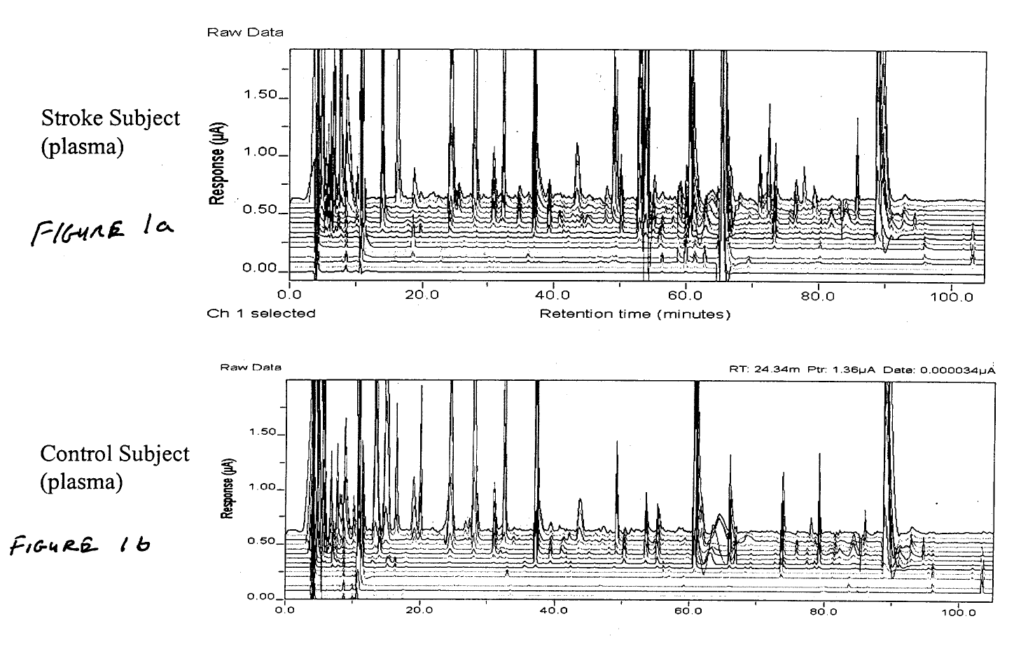

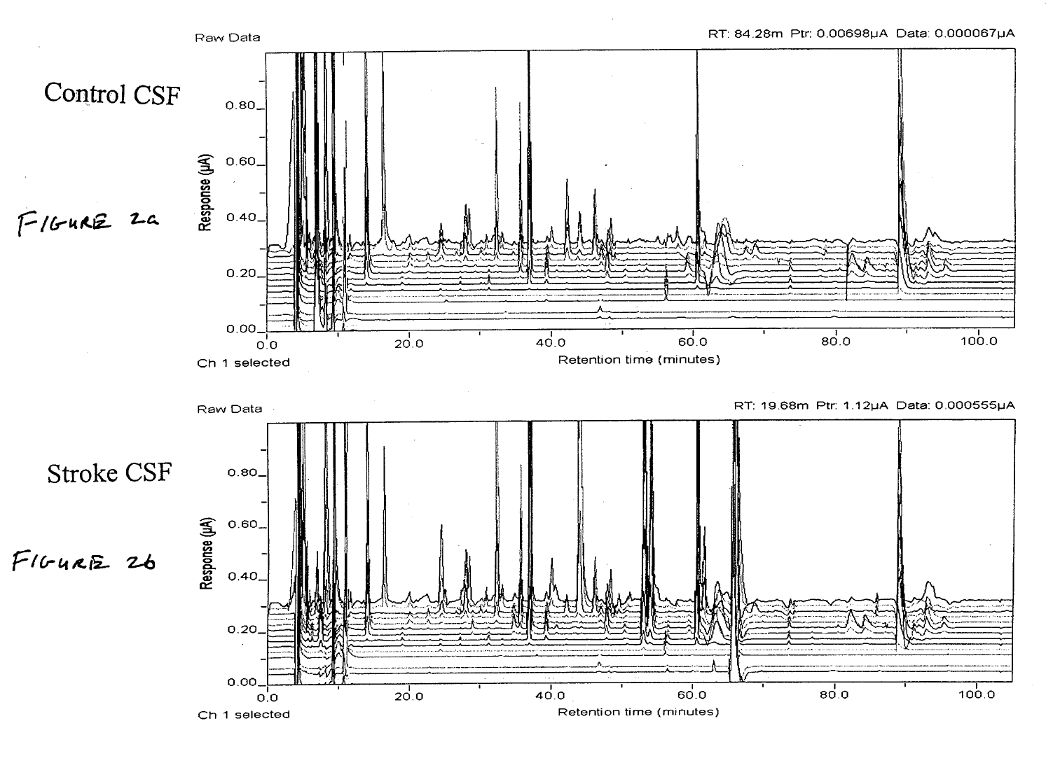

Data from array patterns such as those shown in FIGS. 1a and 1b and 2a and 2b for plasma and CSF, respectively, were subjected to algorithms for peak deconvolution and clustering, and stretched to reference peaks in the profiles. The digitized clusters from pools of control and stroke subjects were then matched to each individual sample. This provided a relative value quantitative table for each of the ca 1600 components isolated which is directly compatible with cluster analysis, frequency distribution analysis or other categorical techniques. It also provided a preliminary search for unique compounds existing in one or the other of the groups.

Results:

The results discussed below are based on the initial data mining of Cohort two, and on ancillary studies of mechanism suggested by the data in Cohorts one and two.

A portion of a data table from the Electrochemical array studies of a subset of the cohort two study is shown in the attached Tables A-AK, AL-BB, BC-B4, BZ-DD and DE-EL. While all of the data has not been mined for all of the correlation and pathway relationships there are several salient features.

There is massive statistically significant reduction of the levels of primary antioxidants Ascorbic acid (in plasma and CSF) and Tocopherol (in plasma).

There are statistically significant shifts in purine pathway relationships, kynurinine pathway relationships and serotonergic pathway relationships.

There are significant increases over normal in 1,3,7, and 8 methyl xanthine and 8 methyl xanthosine.

At day 7-21 there are significant increases in the levels of plasma 7 methyl guanine (7MG).

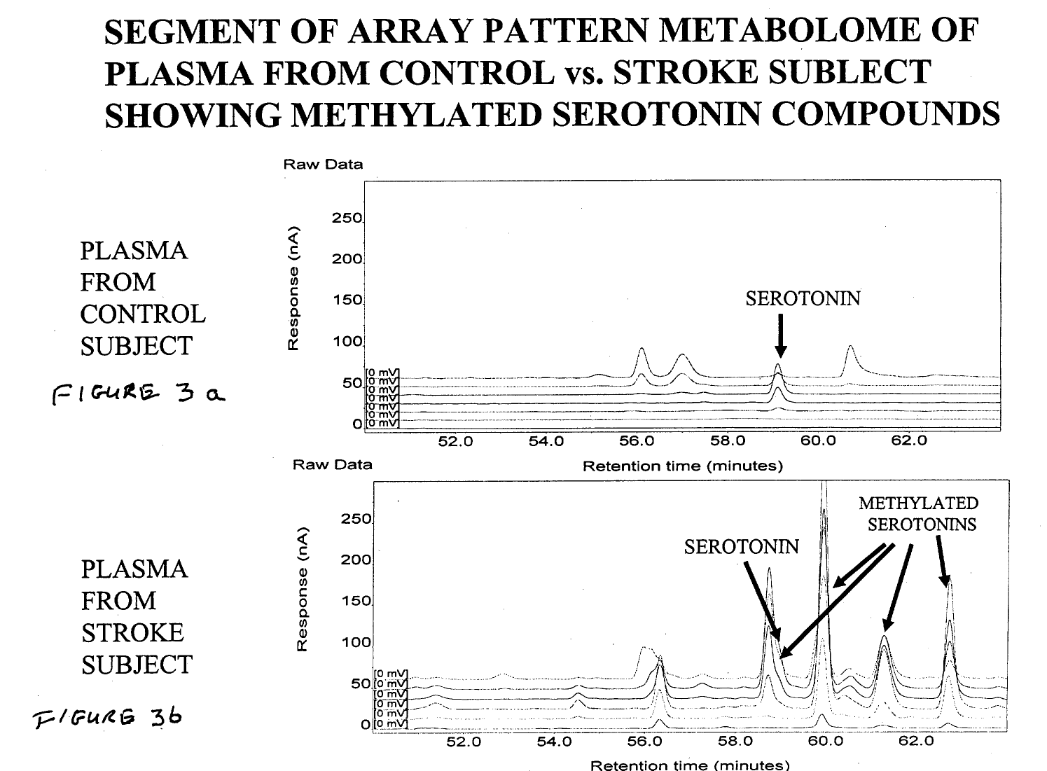

The most striking feature of the profiles of plasma in stroke subjects is the occurrence of methylated forms of serotonin as shown in FIGS. 3a and 3b, representing a portion of the array profile of a stroke subject plasma compared to a control subject. The occurrence of methylated serotonins is accompanied by the occurrence of a peak of unknown identity at array coordinates of 66 min ch3 which occurs in both CSF and plasma, and by the depletion of peaks of unknown identity at array coordinates of 84 min ch4, 86 min ch5 and 96 min ch3.

Methylated compounds and specifically methylated serotonins are uniformly significantly higher in the jugular vein samples and in the stroke side jugular vein.

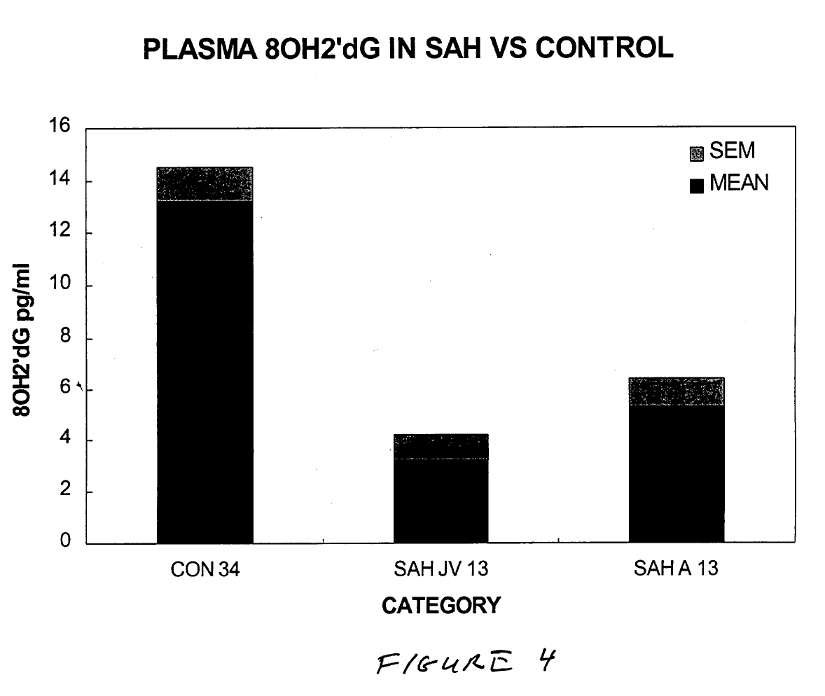

Methylation is accompanied by and correlated with a reduction in the oxidative DNA damage and repair marker 8OH2′dG as shown in FIG. 4. The decrease in this marker is also greater in the jugular vein than in the peripheral samples.

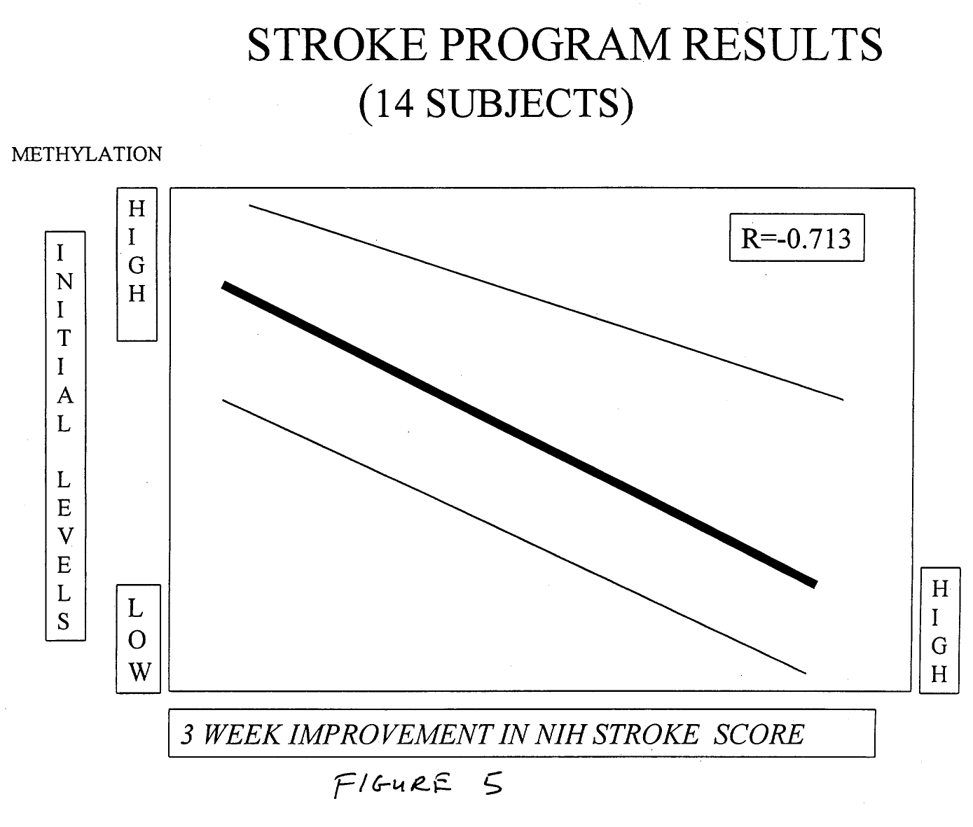

The level of methylation in the initial plasma or CSF samples from a stroke subject predicts the rate of improvement on the NIH stroke scale as shown in FIG. 5.

DNA isolated from the CSF of stroke subjects and control subjects as described above shows a number of significant differences. First it should be noted that in control subjects the levels of DNA as measured by a principle nucleotide level such as 2′deoxyguanosine are ca 5-10 fold lower. This would suggest that DNA found in CSF represents a mechanical process whereby cells or necrotic/apoptotic cell products are being dumped from the CNS faster than the nuclease mechanisms can handle them. This would also suggest that the DNA in CSF from stroke subjects reflects the processes operating in the CNS.

Normalized to 2′dG levels using carbon column switching techniques levels of 7MG in the CSF DNA were ca 8 fold higher and levels of the oxidative damage marker 8OH2′dG were 15% lower but not statistically significant.

While there were several quantitative differences in the overall array patterns of the CSF DNA one striking finding of mechanistic significance was the absence of the peaks described by the array coordinates 84/4, 86/5 and 96/3.

A second finding was the appearance in the stroke CSF DNA of compounds with array coordinates from 68-77 min on ch9 and 10. These are in the region where indoles respond in the array and are possibly methylated, acetylated or hydroxy indoles that are previously undescribed. This class of compounds is not as tightly bound as the peaks at 84/4, 86/5 and 96/3 and could be replaced with other adducts such as adenosine or indole pyruvic acid.

A preparation containing primarily peaks 86/4, 86/5, and 96/3 was isolated from a pool of control plasma. This preparation when added to calf thymus DNA in an invitro study suppressed the rate of oxidative DNA damage in a time damage study more effectively than bovine serum albumin co enzyme Q10, or tocopherol.

Cluster analysis of the data groups as a whole showed a complete separation of control subjects from stroke subjects with both with all variables and with a selected set of as few as 14 variables of the methylation and purine and kynurinine systems. Subjects at 21 days showed a slight separation from subjects at day 1, and complete separation from controls.

Within the stroke group, cluster analysis separated sub-arachnoid hemorrhage from embolic or thrombolytic stroke groups.

The findings from this study indicate that in acute care of stroke increased oxidative damage protection agents in the CNS and modulation of methylation pathways will have a beneficial effect. The absence of endogenous antioxidant compounds in the CNS derived DNA of stroke subjects suggests these compounds or their combinatorial analogs as therapeutic agents. Conversely, those compounds that are present in the DNA of stroke subjects suggest therapies based on replacement with replacement adducts that return the functionality and expression of the genes affected.

The data suggests that there are two areas of stroke therapy, acute and long term. The specific nature of acute care may be driven by rapid assessment of critical markers such as overall methylation that indicate one form of antioxidant and N methyl transferrase suppression, conversely the assessment of specific potentially neurotoxic forms such as NN dimethyl serotonin may indicate the administration of methyl acceptors.

TABLE A-AK

| | 2HPAC | 3OHAN | 3OHKY | 3OMD | 4HBAC | 4HPAC | 4HPLA | 5HIAA | 5HT | 5HTOL | 5HTP | AM |
|---|---|---|---|---|---|---|---|---|---|---|---|---|
| CSF1_I | 0.23 | 0.00 | 0.51 | 4.39 | 1.72 | 14.41 | 68.85 | 34.19 | 0.00 | | 1.17 | |
| CSF1_II | 0.14 | 0.00 | 0.47 | 1.41 | 0.98 | 6.30 | 56.01 | 33.98 | 0.00 | | 1.29 | |
| CSF1_III | 0.00 | 0.00 | 0.53 | 2.26 | 0.51 | 6.87 | 54.66 | 16.92 | 0.00 | | 0.78 | |
| CSF1_IV | 0.49 | 0.00 | 0.46 | 4.24 | 0.49 | 34.00 | 113.97 | 36.70 | 0.00 | | 1.29 | |
| CSF1_V | 1.00 | 0.00 | 0.75 | 3.30 | 0.34 | 6.71 | 50.03 | 21.40 | 0.00 | | 0.83 | |
| CSF3_I | 0.00 | 0.00 | 0.80 | 1.83 | 0.42 | 24.87 | 99.57 | 47.65 | 0.00 | | 0.77 | |
| CSF3_II_ | 0.00 | 0.00 | 0.67 | 5.18 | 0.47 | 23.37 | 133.41 | 58.19 | 0.00 | | 1.35 | |
| CSF3_III | 0.00 | 0.00 | 0.44 | 5.83 | 0.40 | 9.09 | 72.28 | 26.35 | 0.00 | | 0.81 | |
| CSF3_IV | 0.76 | 0.00 | 0.48 | 1.48 | 0.35 | 29.35 | 61.78 | 23.58 | 0.00 | | 0.87 | |
| CSF3_V | 0.00 | 0.00 | 0.64 | 4.25 | 0.36 | 10.48 | 51.76 | 14.72 | 0.00 | | 0.59 | |
| PLASMA_1_I_AD_ | 0.27 | 0.46 | 3.33 | 17.73 | 2.73 | 84.86 | 127.51 | 4.68 | 40.5 | | 0.22 | 6.98 |
| PLASMA_1_I_CVD_ | 0.00 | 0.00 | 2.80 | 16.44 | 3.15 | 92.44 | 166.47 | 4.32 | 20.84 | | 0.08 | |
| PLASMA_1_I_VD_ | 0.00 | 0.00 | 4.00 | 15.22 | 2.58 | 98.37 | 157.38 | 5.46 | 41.22 | | 0.21 | |
| PLASMA_1_I_VS | 0.00 | 0.00 | 3.85 | 17.77 | 3.95 | 95.80 | 139.45 | 5.55 | 39.15 | | 0.25 | |
| PLASMA_1_II_AD | 1.45 | 1.16 | 6.74 | 12.93 | 2.86 | 47.83 | 111.64 | 7.41 | 0.67 | | 0.25 | |
| PLASMA_1_II_CVD | 1.84 | 1.06 | 7.13 | 16.14 | 5.06 | 47.87 | 131.39 | 7.60 | 4.07 | | 0.42 | |
| PLASMA_1_II_VD | 0.99 | 0.00 | 0.00 | 13.32 | 3.09 | 43.07 | 115.00 | 1.57 | 1.39 | | 0.91 | |
| PLASMA_1_II_VS | 1.46 | 2.43 | 7.94 | 14.69 | 7.22 | 47.23 | 125.28 | 8.33 | 1.79 | | 0.53 | |
| PLASMA_1_III_AD | 0.00 | 0.62 | 4.29 | 13.60 | 1.93 | 62.37 | 179.81 | 9.62 | 17.57 | | 0.25 | |
| PLASMA_1_III_AS | 0.00 | 0.84 | 4.18 | 14.53 | 1.64 | 61.61 | 157.71 | 9.70 | 21.80 | | 0.17 | |
| PLASMA_1_III_CVD | 0.00 | 1.38 | 4.05 | 13.42 | 2.13 | 63.04 | 187.59 | 9.95 | 31.48 | | 0.18 | |
| PLASMA_1_III_VD | 3.12 | 0.36 | 3.01 | 12.51 | 2.61 | 64.39 | 143.45 | 8.58 | 1.59 | | 0.12 | |
| PLASMA_1_IV_AD | 5.26 | 2.06 | 2.67 | 14.38 | 2.47 | 249.98 | 301.38 | 7.08 | 33.36 | | 0.15 | |
| PLASMA_1_IV_CVD | 0.00 | 1.33 | 2.35 | 13.03 | 2.25 | 244.75 | 355.96 | 6.81 | 39.88 | | 0.14 | |
| PLASMA_1_IV_VD | 3.50 | 0.35 | 1.69 | 11.58 | 1.73 | 205.85 | 297.25 | 5.99 | 8.60 | | 0.22 | |
| PLASMA_1_IV_VS | 3.69 | 1.72 | 2.57 | 14.01 | 2.10 | 225.32 | 306.01 | 6.73 | 18.55 | | 0.31 | |
| PLASMA_1_V_ARTERIA | 0.00 | 1.24 | 3.82 | 13.79 | 0.77 | 23.43 | 86.68 | 11.87 | 187.39 | | 0.36 | |
| PLASMA_1_V_CVD | 0.00 | 1.40 | 3.82 | 13.82 | 0.53 | 21.11 | 97.72 | 10.79 | 101.89 | | 0.23 | |
| PLASMA_1_V_V.SINISTRA | 0.00 | 1.30 | 4.15 | 14.22 | 0.82 | 21.23 | 91.97 | 11.29 | 171.60 | | 0.13 | |
| PLASMA_1_V_V.DEXTRA | 0.00 | 1.45 | 4.08 | 14.33 | 1.03 | 23.06 | 101.07 | 11.77 | 279.06 | | 0.23 | |
| PLASMA_3_I_AD | 0.00 | 1.46 | 3.28 | 13.43 | 2.47 | 121.26 | 158.29 | 6.23 | 45.92 | | 0.15 | |
| PLASMA_3_I_CVD | 0.00 | 1.27 | 2.60 | 15.29 | 1.98 | 142.95 | 255.82 | 6.10 | 8.87 | | 0.09 | |
| PLASMA_3_I_VD | 0.00 | 1.68 | 2.15 | 15.12 | 1.16 | 122.29 | 183.35 | 5.53 | 2.32 | | 0.19 | |
| PLASMA_3_I_VS | 0.00 | 2.19 | 3.19 | 14.72 | 2.56 | 135.95 | 199.80 | 7.74 | 45.67 | | 0.33 | |
| PLASMA_3_II_AS | 1.81 | 0.86 | 2.31 | 16.03 | 1.20 | 111.63 | 188.70 | 4.75 | 1.36 | | 0.20 | |
| PLASMA_3_II_CVD | 1.99 | 0.36 | 1.23 | 13.65 | 1.12 | 98.16 | 214.04 | 4.05 | 0.36 | | 0.23 | |
| PLASMA_3_II_VD | 1.68 | 0.68 | 2.72 | 14.80 | 1.68 | 96.51 | 182.77 | 3.97 | 0.39 | | 0.32 | |
| PLASMA_3_II_VS | 1.90 | 1.10 | 1.62 | 14.09 | 2.53 | 101.98 | 191.94 | 4.22 | 0.39 | | 0.30 | |
| PLASMA_3_III_AD | 0.00 | 0.06 | 3.94 | 19.46 | 2.29 | 70.77 | 200.08 | 11.28 | 34.73 | | 0.35 | |
| PLASMA_3_III_CVD | 0.00 | 1.27 | 4.74 | 20.47 | 1.21 | 77.15 | 257.67 | 12.47 | 3.17 | | 0.37 | |
| PLASMA_3_III_VD | 0.00 | 0.00 | 3.78 | 20.27 | 2.09 | 75.93 | 209.45 | 12.01 | 30.68 | | 0.35 | |
| PLASMA_3_III_VS | 0.00 | 1.19 | 4.88 | 18.41 | 5.59 | 72.07 | 208.57 | 12.15 | 6.88 | | 0.19 | |
| PLASMA_3_IV_AD | 0.00 | 0.69 | 2.84 | 12.37 | 2.15 | 530.53 | 235.52 | 6.13 | 30.82 | | 0.17 | |
| PLASMA_3_IV_CVD | 0.00 | 1.93 | 3.14 | 14.53 | 3.18 | 579.73 | 285.01 | 6.19 | 37.87 | | 0.11 | |
| PLASMA_3_IV_VD | 0.00 | 1.87 | 3.22 | 13.14 | 4.28 | 552.15 | 266.34 | 6.68 | 36.98 | | 0.12 | |
| PLASMA_3_IV_VS | 0.00 | 1.44 | 3.26 | 11.47 | 2.98 | 564.80 | 281.00 | 6.93 | 61.40 | | 0.21 | |
| PLASMA_3_V_ARTERIA | 41.83 | 2.36 | 6.06 | 12.24 | 2.09 | 90.25 | 213.11 | 9.54 | 26.96 | | 0.35 | |
| PLASMA_3_V_CVD | 35.48 | 1.33 | 5.54 | 12.31 | 1.57 | 75.54 | 283.90 | 8.39 | 40.07 | | 0.15 | |
| PLASMA_3_V_VDEXTRA | 40.35 | 2.48 | 5.71 | 11.33 | 1.73 | 82.62 | 218.32 | 9.98 | 128.63 | | 0.28 | |
| PLASMA_3_V_VSINISTRA | 48.59 | 2.42 | 5.98 | 11.79 | 3.71 | 87.40 | 226.68 | 10.44 | 88.40 | | 0.36 | |
| PLASMA_7_I_CVD | 0.00 | 0.00 | 4.22 | 10.72 | 4.62 | 103.90 | 259.90 | 5.28 | 48.22 | | 0.24 | |
| PLASMA_7_II_CVD | 5.08 | 0.68 | 7.63 | 18.06 | 4.32 | 490.92 | 410.07 | 6.14 | 3.21 | | 0.20 | |
| PLASMA_7_III_CVD | 5.06 | 2.02 | 9.04 | 16.80 | 3.36 | 160.79 | 374.55 | 11.82 | 31.61 | | 0.29 | |
| PLASMA_7_IV_CVD | 14.18 | 2.81 | 3.52 | 18.50 | 5.97 | 267.28 | 286.07 | 8.05 | 19.73 | | 0.08 | |
| PLASMA_7_V_CVD | 0.00 | 2.78 | 5.69 | 12.87 | 2.97 | 96.78 | 226.13 | 15.57 | 203.53 | | 0.35 | |
| PLASMA_14_I_CVD | 0.00 | 1.21 | 3.48 | 15.81 | 3.38 | 124.98 | 433.85 | 9.01 | 10.77 | | 0.26 | |
| PLASMA_14_II_CVD | 0.00 | 0.46 | 6.85 | 26.75 | 8.71 | 287.03 | 365.78 | 13.01 | 1.04 | | 0.35 | |
| PLASMA_14_III_CVD | 0.00 | 0.93 | 5.15 | 23.01 | 2.93 | 52.74 | 267.20 | 10.41 | 22.88 | | 0.26 | |
| PLASMA_14_IV_CVD | 0.00 | 2.01 | 4.27 | 15.81 | 3.89 | 221.41 | 246.97 | 7.17 | 8.07 | | 0.16 | |
| PLASMA_21_I_CVD | 47.54 | 0.00 | 5.65 | 22.84 | 9.69 | 353.06 | 450.85 | 9.22 | 4.98 | | 0.19 | |
| PLASMA_21_II_CVD | 97.38 | 0.69 | 9.44 | 32.79 | 0.00 | 4266.75 | 911.92 | 29.62 | 2.98 | | 0.60 | |
| PLASMA_21_III | 1.74 | 1.41 | 4.68 | 25.09 | 4.47 | 206.42 | 268.47 | 17.27 | 72.62 | | 0.30 | |
| PLASMA_21_IV_CVD | 4.01 | 3.46 | 3.24 | 14.45 | 2.97 | 228.99 | 186.14 | 5.31 | 1.67 | | 0.13 | |
| 1S_ | 0.00 | 2.54 | 10.03 | 24.64 | 0.00 | 77.82 | 192.87 | 15.27 | 148.69 | 0.25 | 1.09 | 139.54 |
| 1A_ | 4.81 | 0.00 | 9.55 | 24.65 | 0.00 | 80.79 | 197.83 | 12.37 | 246.49 | | 1.23 | 140.09 |
| 1D_ | 4.78 | 4.74 | 10.00 | 25.75 | 0.00 | 85.85 | 209.31 | 11.39 | 64.68 | | 0.56 | 152.50 |
| 2S_ | 8.65 | 3.46 | 7.22 | 17.42 | 0.00 | 115.57 | 267.61 | 11.56 | 208.82 | | 0.21 | 89.13 |
| 2A_ | 8.77 | 4.84 | 5.49 | 18.15 | 0.00 | 116.16 | 271.07 | 11.24 | 125.09 | | 0.36 | 92.32 |
| 2D_ | 0.00 | 0.00 | 7.02 | 16.07 | 0.00 | 112.38 | 255.78 | 10.05 | 113.56 | | 0.32 | 84.35 |
| 3S_ | 30.34 | 5.89 | 12.21 | 21.63 | 0.00 | 532.68 | 457.40 | 15.63 | 12.73 | | 0.79 | 122.37 |
| 3A_ | 26.57 | 4.89 | 11.05 | 20.82 | 0.00 | 485.20 | 445.74 | 13.68 | 74.56 | | 0.69 | 112.66 |
| 3D_ | 27.99 | 5.26 | 11.02 | 15.76 | 0.00 | 502.45 | 399.52 | 13.54 | 17.89 | | 0.49 | 117.40 |
| 4S_ | 1.64 | 0.00 | 10.51 | 24.12 | 0.00 | 77.71 | 207.13 | 10.62 | 64.53 | | 0.35 | 86.10 |
| 4A_ | 0.00 | 6.04 | 9.85 | 22.46 | 0.00 | 86.62 | 197.81 | 9.27 | 14.52 | | 0.78 | |
| 4D_ | 0.00 | 7.12 | 9.89 | 21.44 | 0.00 | 77.93 | 206.33 | 9.49 | 37.93 | | 0.58 | |
| 5S_ | 0.00 | 0.81 | 4.27 | 9.27 | 0.00 | 75.18 | 271.95 | 6.51 | 24.59 | | 0.77 | 34.05 |
| 5A_ | 0.00 | 1.27 | 5.63 | 9.75 | 0.00 | 80.94 | 285.45 | 7.00 | 38.92 | | 0.54 | |

| | ASC | CYC | DA** | DOPAC | EPI | G | GR | GSH | GSSG | HGA | HVA | HX |
|---|---|---|---|---|---|---|---|---|---|---|---|---|
| CSF1_I | 0.00 | | 17.87 | | 0.02 | 0.00 | 13.80 | | 0.00 | 0.00 | 57.18 | 583.50 |
| CSF1_II | 0.00 | | 17.63 | | 0.02 | 0.00 | 11.29 | | 0.00 | 0.00 | 49.69 | 119.29 |
| CSF1_III | 0.00 | | 20.04 | | 0.02 | 0.00 | 9.14 | | 0.00 | 0.00 | 26.55 | 339.86 |
| CSF1_IV | 0.00 | | 13.59 | | 0.02 | 0.00 | 9.79 | | 0.00 | 0.00 | 53.11 | 310.74 |
| CSF1_V | 5798.78 | | 19.92 | | 0.02 | 0.00 | 11.02 | | 0.00 | 0.00 | 32.82 | 776.66 |
| CSF3_I | 4.92 | | 18.35 | | 0.02 | 0.00 | 6.41 | | 0.00 | 0.00 | 109.35 | 182.94 |
| CSF3_II_ | 100.03 | | 14.25 | | 0.02 | 0.00 | 6.87 | | 0.00 | 0.00 | 106.27 | 154.07 |
| CSF3_III | 0.00 | | 18.27 | | 0.02 | 0.00 | 7.55 | | 0.00 | 0.00 | 31.99 | 480.90 |
| CSF3_IV | 0.00 | | 17.76 | | 0.02 | 0.00 | 10.94 | | 0.00 | 0.00 | 32.95 | 362.98 |
| CSF3_V | 10420.11 | | 18.14 | | 0.02 | 0.00 | 2.36 | | 0.00 | 0.00 | 30.72 | 89.09 |
| PLASMA_1_I_AD_ | 1.24 | | 0 | 0.15 | 9.77 | 0.77 | 4.43 | 0.32 | 19.2 | 0.01 | 11.09 | 1239.33 |
| PLASMA_1_I_CVD_ | 0.00 | | 1.72 | | 0.04 | 0.00 | 18.30 | | 11.90 | 0.00 | 11.73 | 859.81 |
| PLASMA_1_I_VD_ | 0.00 | | 0.11 | | 0.04 | 0.00 | 4.74 | | 19.16 | 0.00 | 12.69 | 1059.18 |
| PLASMA_1_I_VS | 0.00 | | 1.64 | | 0.04 | 0.00 | 1.70 | | 22.65 | 0.00 | 14.33 | 804.67 |
| PLASMA_1_II_AD | 0.00 | | 2.03 | | 0.04 | 0.45 | 12.80 | | 28.13 | 0.00 | 23.22 | 1852.76 |
| PLASMA_1_II_CVD | 0.00 | | 5.47 | | 0.04 | 1.40 | 43.92 | | 28.72 | 0.00 | 24.36 | 907.21 |
| PLASMA_1_II_VD | 0.00 | | 5.17 | | 0.04 | 0.84 | 33.07 | | 19.09 | 0.00 | 22.20 | 340.90 |
| PLASMA_1_II_VS | 0.00 | | 5.43 | | 0.04 | 0.41 | 10.25 | | 12.91 | 0.00 | 24.41 | 641.44 |
| PLASMA_1_III_AD | 0.00 | | 7.46 | | 0.04 | 0.90 | 28.34 | | 6.09 | 0.00 | 24.85 | 1112.36 |
| PLASMA_1_III_AS | 0.00 | | 6.22 | | 0.04 | 0.00 | 4.82 | | 41.51 | 0.00 | 21.45 | 1271.94 |
| PLASMA_1_III_CVD | 0.00 | | 6.19 | | 0.04 | 0.64 | 65.95 | | 46.34 | 0.00 | 21.73 | 2012.91 |
| PLASMA_1_III_VD | 0.00 | | 6.39 | | 0.04 | 0.31 | 34.61 | | 31.60 | 0.00 | 20.37 | 569.13 |
| PLASMA_1_IV_AD | 0.00 | | 5.80 | | 0.04 | 0.70 | 56.28 | | 31.88 | 0.00 | 15.21 | 1359.38 |
| PLASMA_1_IV_CVD | 0.00 | | 5.10 | | 0.04 | 0.00 | 16.47 | | 29.36 | 0.00 | 15.56 | 1138.60 |
| PLASMA_1_IV_VD | 0.00 | | 4.73 | | 0.04 | 0.00 | 6.30 | | 40.10 | 0.00 | 13.66 | 165.14 |
| PLASMA_1_IV_VS | 0.00 | | 5.86 | | 0.04 | 0.00 | 2.86 | | 49.90 | 0.00 | 15.32 | 229.69 |
| PLASMA_1_V_ARTERIA | 0.00 | | 4.30 | | | 4.73 | 72.26 | | 17.14 | 0.65 | 7.84 | 2224.89 |
| PLASMA_1_V_CVD | 0.00 | | 1.51 | | | 2.53 | 31.49 | | 43.96 | 0.64 | 7.85 | 1674.72 |
| PLASMA_1_V_V.SINISTRA | 0.00 | | 3.95 | | | 1.55 | 33.49 | | 39.36 | 0.44 | 8.94 | 1119.25 |
| PLASMA_1_V_V.DEXTRA | 0.00 | | 1.30 | | 0.04 | 1.76 | 32.69 | | 23.54 | 0.47 | 8.65 | 1396.36 |
| PLASMA_3_I_AD | 0.00 | | 4.74 | | 0.04 | 1.19 | 27.49 | | 78.97 | 0.00 | 19.31 | 2364.48 |
| PLASMA_3_I_CVD | 0.00 | | 4.74 | | 0.04 | 0.58 | 37.63 | | 73.87 | 0.00 | 22.53 | 1084.89 |
| PLASMA_3_I_VD | 0.00 | | 6.31 | | 0.04 | 0.40 | 4.67 | | 79.72 | 0.00 | 20.16 | 475.57 |
| PLASMA_3_I_VS | 0.00 | | 6.08 | | 0.04 | 1.55 | 43.51 | | 78.16 | 0.00 | 23.36 | 2549.67 |
| PLASMA_3_II_AS | 0.00 | | 5.77 | | 0.04 | 0.00 | 31.89 | | 51.04 | 0.00 | 20.73 | 1384.10 |
| PLASMA_3_II_CVD | 0.00 | | 5.60 | | 0.04 | 0.00 | 33.33 | | 74.34 | 0.00 | 19.45 | 487.19 |
| PLASMA_3_II_VD | 0.00 | | 6.60 | | 0.04 | 0.00 | 22.54 | | 77.30 | 0.00 | 19.77 | 546.88 |
| PLASMA_3_II_VS | 0.00 | | 6.60 | | 0.04 | 0.00 | 21.73 | | 98.77 | 0.00 | 20.84 | 374.64 |
| PLASMA_3_III_AD | 0.00 | | 8.27 | | 0.04 | 0.68 | 67.82 | | 80.70 | 0.00 | 35.30 | 1812.64 |
| PLASMA_3_III_CVD | 0.00 | | 10.31 | | 0.04 | 0.00 | 48.79 | | 95.26 | 0.00 | 37.94 | 345.48 |
| PLASMA_3_III_VD | 0.00 | | 8.45 | | 0.04 | 0.79 | 118.79 | | 103.73 | 0.00 | 36.38 | 1206.62 |
| PLASMA_3_III_VS | 0.00 | | 7.37 | | 0.04 | 0.74 | 27.36 | | 84.07 | 0.00 | 38.87 | 435.39 |
| PLASMA_3_IV_AD | 0.00 | | 4.90 | | 0.04 | 0.37 | 6.70 | | 51.52 | 0.00 | 15.81 | 1876.02 |
| PLASMA_3_IV_CVD | 0.00 | | 3.28 | | 0.04 | 0.00 | 27.89 | | 102.16 | 0.00 | 15.79 | 1690.50 |
| PLASMA_3_IV_VD | 0.00 | | 3.91 | | 0.04 | 0.00 | 4.20 | | 119.75 | 0.00 | 19.12 | 1243.23 |
| PLASMA_3_IV_VS | 0.00 | | 0.95 | | 0.04 | 0.00 | 3.05 | | 106.45 | 0.00 | 18.04 | 1152.09 |
| PLASMA_3_V_ARTERIA | 0.00 | | 3.70 | | 0.04 | 0.00 | 65.33 | | 73.04 | 0.99 | 15.31 | 690.31 |
| PLASMA_3_V_CVD | 0.00 | | 5.03 | | 0.04 | 0.00 | 62.31 | | 98.95 | 0.89 | 13.11 | 643.45 |
| PLASMA_3_V_VDEXTRA | 0.00 | | 3.93 | | 0.04 | 2.78 | 108.27 | | 116.62 | 1.12 | 13.99 | 1233.36 |
| PLASMA_3_V_VSINISTRA | 0.00 | | 4.90 | | 0.04 | 0.00 | 80.67 | | 84.17 | 1.10 | 15.03 | 1068.70 |
| PLASMA_7_I_CVD | 0.00 | | 4.26 | | 0.04 | 0.62 | 25.07 | | 110.42 | 0.00 | 20.33 | 1562.24 |
| PLASMA_7_II_CVD | 0.00 | | 4.42 | | 0.04 | 0.00 | 19.13 | | 168.66 | 0.00 | 36.76 | 256.40 |
| PLASMA_7_III_CVD | 0.00 | | 3.81 | | 0.04 | 0.00 | 10.08 | | 172.23 | 0.00 | 53.19 | 1436.51 |
| PLASMA_7_IV_CVD | 0.00 | | 3.73 | | 0.04 | 0.00 | 1.82 | | 124.08 | 0.00 | 16.29 | 440.86 |
| PLASMA_7_V_CVD | 0.00 | | 3.64 | | 0.04 | 1.48 | 74.89 | | 76.16 | 3.31 | 32.28 | 976.01 |
| PLASMA_14_I_CVD | 0.00 | | 4.75 | | 0.04 | 0.00 | 74.77 | | 164.41 | 0.00 | 44.03 | 676.08 |
| PLASMA_14_II_CVD | 0.00 | | 1.51 | | 0.04 | 0.00 | 8.45 | | 178.26 | 0.00 | 70.52 | 99.98 |
| PLASMA_14_III_CVD | 0.00 | | 3.19 | | 0.04 | 0.00 | 8.40 | | 140.29 | 0.00 | 33.73 | 225.95 |
| PLASMA_14_IV_CVD | 0.00 | | 3.05 | | 0.04 | 0.00 | 20.80 | | 98.28 | 0.00 | 27.33 | 1171.69 |
| PLASMA_21_I_CVD | 0.00 | | 3.57 | | 0.04 | 0.00 | 7.58 | | 178.07 | 0.00 | 145.04 | 809.52 |
| PLASMA_21_II_CVD | 0.00 | | 11.74 | | | 0.00 | 3.77 | | 344.33 | 0.00 | 221.90 | 58.14 |
| PLASMA_21_III | 0.00 | | 0.32 | | | 1.04 | 27.87 | | 111.43 | 0.00 | 85.65 | 1721.57 |
| PLASMA_21_IV_CVD | 0.00 | | 7.15 | | | 1.38 | 47.18 | | 129.45 | 0.00 | 23.47 | 1260.91 |
| 1S_ | 5.57 | 520.12 | 16.50 | | | | 29.82 | 38.71 | 13.97 | 5.53 | 25.24 | 2535.43 |
| 1A_ | 6.62 | 509.81 | 0.00 | | | | 36.20 | | 15.13 | 5.38 | 23.35 | 3691.93 |
| 1D_ | 5.00 | 538.72 | 3.74 | | | | 30.47 | | 18.20 | 3.48 | 24.23 | 2070.41 |
| 2S_ | 6.93 | 507.73 | 10.09 | | | | 23.10 | 38.89 | 11.17 | 0.01 | 31.31 | 2379.28 |
| 2A_ | 8.32 | 512.61 | 11.33 | | | | 17.13 | | 11.90 | 0.01 | 31.56 | 2144.91 |
| 2D_ | 6.21 | 496.73 | 0.00 | | | | 9.23 | | 21.39 | 0.01 | 29.96 | 1464.01 |
| 3S_ | 11.65 | 692.81 | 6.23 | 4.12 | | | 2.29 | | 0.00 | 0.01 | 94.46 | 298.43 |
| 3A_ | 7.57 | 675.44 | 8.42 | 3.46 | | | 9.93 | | 21.91 | 0.01 | 83.91 | 903.43 |
| 3D_ | 5.07 | 676.57 | 5.64 | 2.91 | | | 7.92 | | 36.70 | 0.01 | 85.27 | 402.11 |
| 4S_ | 4.03 | 543.94 | 19.86 | | | | 5.98 | | 10.68 | 4.25 | 23.09 | 1085.15 |
| 4A_ | 4.73 | 478.76 | 3.65 | | | | 2.53 | | 8.47 | 0.01 | 19.69 | 330.27 |
| 4D_ | 4.75 | 497.02 | 4.29 | | | | 9.08 | | 3.81 | 5.77 | 21.16 | 687.58 |
| 5S_ | 1.03 | 492.84 | 1.53 | | | | 4.29 | | 0.00 | 0.01 | 16.49 | 1357.93 |
| 5A_ | 4.36 | 496.66 | 5.14 | 0.86 | | | 3.85 | | 7.90 | 0.01 | 16.94 | 1120.35 |
| | KYN | LD | MEL | MET | MHPG | TPOL | TRP | TRYPT | TYR | TYRA | NA5HT | NE |
| CSF1_I | 11.15 | | 0.33 | 392.86 | 19.16 | | 443.10 | | 2009.17 | | | |
| CSF1_II | 14.70 | | 0.17 | 421.20 | 11.68 | | 903.77 | | 1311.05 | | | |
| CSF1_III | 9.33 | | 0.25 | 575.60 | 15.10 | | 372.10 | | 1510.44 | | | |
| CSF1_IV | 9.78 | | 0.50 | 824.12 | 18.80 | | 631.97 | | 3252.10 | | | |
| CSF1_V | 8.33 | | 0.42 | 549.45 | 12.94 | | 359.85 | | 1123.21 | | | |
| CSF3_I | 14.38 | | 0.32 | 570.00 | 25.04 | | 417.12 | | 1663.61 | | | |
| CSF3_II_ | 11.91 | | 0.00 | 544.52 | 18.33 | | 553.99 | | 1775.61 | | | |
| CSF3_III | 11.81 | | 0.21 | 541.89 | 18.33 | | 325.43 | | 1515.21 | | | |
| CSF3_IV | 8.54 | | 0.46 | 577.09 | 15.93 | | 473.62 | | 2116.87 | | | |
| CSF3_V | 11.05 | | 0.00 | 397.88 | 18.32 | | 329.09 | | 1098.49 | | | |
| PLASMA_1_I_AD_ | 290.5 | 1.51 | 4.28 | 2275.28 | 10.75 | | 5948.0 | 43.29 | 5871.4 | 0.97 | 1.15 | |
| PLASMA_1_I_CVD_ | 320.72 | | 9.18 | 2432.20 | 11.68 | | 6098.8 | | 5870.8 | | | |
| PLASMA_1_I_VD_ | 375.53 | | 0.00 | 2670.00 | 12.51 | | 7200.0 | | 6403.1 | | | |
| PLASMA_1_I_VS | 355.00 | | 6.24 | 2460.51 | 12.48 | | 6851.4 | | 6846.2 | | | |
| PLASMA_1_II_AD | 421.67 | | 0.00 | 2875.50 | 7.79 | | 11301.2 | | 6983.4 | | | |
| PLASMA_1_II_CVD | 443.77 | | 0.69 | 3191.15 | 8.78 | | 11022.5 | | 7336.0 | | | |
| PLASMA_1_II_VD | 409.13 | | 0.98 | 2964.12 | 8.44 | | 10076.9 | | 7046.2 | | | |
| PLASMA_1_II_VS | 398.07 | | 3.70 | 2778.73 | 8.68 | | 12060.7 | | 7122.5 | | | |
| PLASMA_1_III_AD | 290.60 | | 8.58 | 2631.66 | 7.98 | | 6353.8 | | 6321.2 | | | |
| PLASMA_1_III_AS | 255.09 | | 9.62 | 2623.39 | 7.65 | | 6167.6 | | 6513.8 | | | |
| PLASMA_1_III_CVD | 342.13 | | 9.99 | 3041.91 | 8.56 | | 7513.9 | | 7024.0 | | | |
| PLASMA_1_III_VD | 245.44 | | 7.41 | 3733.12 | 6.65 | | 6341.0 | | 7392.6 | | | |
| PLASMA_1_IV_AD | 263.42 | | 2.13 | 3267.55 | 7.39 | | 8831.2 | | 10036.3 | | | |
| PLASMA_1_IV_CVD | 314.83 | | 0.54 | 3637.21 | 8.76 | | 9753.8 | | 10419.7 | | | |
| PLASMA_1_IV_VD | 245.49 | | 0.54 | 3313.26 | 7.95 | | 8019.1 | | 9603.7 | | | |
| PLASMA_1_IV_VS | 255.63 | | 0.75 | 3376.09 | 8.02 | | 8235.8 | | 9728.6 | | | |
| PLASMA_1_V_ARTERIA | 228.98 | | 0.28 | 2826.51 | 4.79 | | 5101.7 | | 4300.9 | | | |
| PLASMA_1_V_CVD | 253.12 | | 1.01 | 2828.99 | 5.67 | | 5205.2 | | 4236.3 | | | |
| PLASMA_1_V_V.SINISTRA | 274.31 | | 0.95 | 2795.28 | 5.57 | | 5558.4 | | 4216.0 | | | |
| PLASMA_1_V_V.DEXTRA | 298.79 | | 0.72 | 2773.82 | 6.02 | | 6189.0 | | 4174.2 | | | |
| PLASMA_3_I_AD | 216.90 | | 13.58 | 3658.20 | 12.07 | | 7615.0 | | 9035.7 | | | |
| PLASMA_3_I_CVD | 277.07 | | 11.70 | 4281.88 | 18.83 | | 8638.7 | | 96.27.1 | | | |
| PLASMA_3_I_VD | 211.09 | | 5.73 | 3429.58 | 14.29 | | 6534.7 | | 8182.2 | | | |
| PLASMA_3_I_VS | 264.13 | | 19.69 | 4146.96 | 15.69 | | 8558.4 | | 9864.6 | | | |
| PLASMA_3_II_AS | 222.00 | | 3.46 | 3396.87 | 9.09 | | 9585.0 | | 8627.1 | | | |
| PLASMA_3_II_CVD | 203.74 | | 0.94 | 3717.25 | 9.85 | | 8408.1 | | 8544.0 | | | |
| PLASMA_3_II_VD | 187.57 | | 2.56 | 3366.44 | 8.99 | | 7519.7 | | 8161.2 | | | |
| PLASMA_3_II_VS | 197.97 | | 2.18 | 3017.01 | 9.37 | | 8180.3 | | 8214.8 | | | |
| PLASMA_3_III_AD | 295.68 | | 4.47 | 3138.73 | 8.71 | | 5718.5 | | 6309.5 | | | |
| PLASMA_3_III_CVD | 352.88 | | 2.40 | 3185.19 | 10.58 | | 6578.0 | | 6821.5 | | | |
| PLASMA_3_III_VD | 318.03 | | 3.80 | 2864.03 | 9.07 | | 6091.9 | | 6399.4 | | | |
| PLASMA_3_III_VS | 305.42 | | 3.94 | 3012.89 | 10.43 | | 5401.7 | | 5887.4 | | | |
| PLASMA_3_IV_AD | 270.32 | | 0.79 | 2860.25 | 7.26 | | 8215.6 | | 8128.0 | | | |
| PLASMA_3_IV_CVD | 329.45 | | 1.17 | 2909.96 | 9.68 | | 9111.6 | | 8051.7 | | | |
| PLASMA_3_IV_VD | 284.28 | | 1.07 | 2999.24 | 8.95 | | 8099.4 | | 8422.2 | | | |
| PLASMA_3_IV_VS | 296.21 | | 4.34 | 3215.74 | 9.59 | | 8463.6 | | 9016.0 | | | |
| PLASMA_3_V_ARTERIA | 272.73 | | 3.14 | 2586.44 | 7.71 | | 6700.6 | | 4864.0 | | | |
| PLASMA_3_V_CVD | 289.74 | | 2.72 | 3158.71 | 9.43 | | 6438.7 | | 5281.2 | | | |
| PLASMA_3_V_VDEXTRA | 274.57 | | 3.08 | 2701.33 | 7.95 | | 6643.4 | | 5014.2 | | | |
| PLASMA_3_V_VSINISTRA | 292.80 | | 3.35 | 2622.94 | 8.21 | | 7018.5 | | 5036.3 | | | |
| PLASMA_7_I_CVD | 297.18 | | 10.82 | 4754.12 | 16.45 | | 7365.3 | | 10686.8 | | | |
| PLASMA_7_II_CVD | 405.39 | | 5.64 | 5663.19 | 13.68 | | 8442.8 | | 12694.2 | | | |
| PLASMA_7_III_CVD | 487.20 | | 10.91 | 4091.70 | 9.65 | | 7448.0 | | 8296.0 | | | |
| PLASMA_7_IV_CVD | 292.57 | | 4.15 | 4377.24 | 6.46 | | 8516.8 | | 10510.2 | | | |
| PLASMA_7_V_CVD | 356.07 | | 0.77 | 3847.56 | 7.10 | | 7927.7 | | 6967.4 | | | |
| PLASMA_14_I_CVD | 357.33 | | 3.15 | 6128.08 | 22.56 | | 7127.7 | | 10925.5 | | | |
| PLASMA_14_II_CVD | 534.98 | | 4.42 | 4997.80 | 16.43 | | 9395.4 | | 11633.2 | | | |
| PLASMA_14_III_CVD | 390.92 | | 3.30 | 3246.55 | 9.70 | | 5676.9 | | 6040.0 | | | |
| PLASMA_14_IV_CVD | 397.59 | | 0.88 | 3931.33 | 5.96 | | 7794.8 | | 11309.5 | | | |
| PLASMA_21_I_CVD | 622.46 | | 0.00 | 4464.19 | 29.05 | | 6140.5 | | 9808.6 | | | |
| PLASMA_21_II_CVD | 1121.60 | | 0.00 | 4204.11 | 26.07 | | 6508.7 | | 8614.8 | | | |
| PLASMA_21_III | 404.94 | | 1.43 | 3474.04 | 12.65 | | 6248.0 | | 6191.4 | | | |
| PLASMA_21_IV_CVD | 333.61 | | 1.96 | 2988.73 | 4.24 | | 6694.2 | | 8729.8 | | | |
| 1S_ | 614.81 | | 0.00 | 3631.65 | 7.45 | 15.25 | 8036.4 | 3.60 | 7897.2 | | | |
| 1A_ | 606.48 | | 0.00 | 4061.15 | 6.75 | 15.24 | 8215.0 | 3.27 | 8673.2 | | | |
| 1D_ | 643.04 | 2.82 | 0.00 | 4206.86 | 7.05 | 13.60 | 8731.2 | 4.07 | 9115.1 | | | |
| 2S_ | 401.71 | | 0.00 | 5460.80 | 6.63 | 21.75 | 8712.7 | 2.50 | 11308.9 | | 0.35 | |
| 2A_ | 412.40 | 7.25 | 0.00 | 5757.98 | 6.67 | 27.76 | 9399.4 | 2.35 | 12104.0 | | 0.71 | |
| 2D_ | 397.76 | | 0.00 | 4927.41 | 6.17 | 27.47 | 8463.6 | 2.24 | 10625.8 | | | |
| 3S_ | 514.11 | 4.69 | 0.92 | 3802.99 | 7.52 | 25.10 | 7213.3 | 0.11 | 8123.1 | | | |
| 3A_ | 468.89 | | 1.66 | 4057.69 | 6.94 | 33.89 | 6934.7 | 0.16 | 8329.2 | | | |
| 3D_ | 450.44 | | 1.69 | 3251.63 | 5.81 | 33.75 | 6435.8 | 0.72 | 7276.3 | | | |
| 4S_ | 750.85 | | 0.00 | 3223.00 | 6.67 | 58.82 | 10531.2 | 2.77 | 9156.3 | | | |
| 4A_ | 822.50 | | 0.00 | 3056.09 | 5.69 | 16.31 | 10262.4 | 2.52 | 8780.3 | | | |
| 4D_ | 849.50 | | 0.00 | 4194.43 | 5.18 | 17.09 | 10604.6 | 2.98 | 9884.9 | | | |
| 5S_ | 219.36 | | 0.00 | 2456.25 | 8.16 | 11.23 | 2850.9 | 2.97 | 4346.5 | | | |
| 5A_ | 225.85 | 2.71 | 0.00 | 2727.30 | 8.33 | 12.70 | 2982.1 | 2.96 | 4899.7 | 2.43 | | |
| 5D_ | 0.00 | 1.38 | 5.32 | 10.13 | 0.00 | 82.97 | 303.42 | 7.35 | 35.69 | | 0.87 | |
| 6S_ | 0.00 | 4.92 | 7.67 | 15.26 | 0.00 | 86.18 | 255.89 | 11.71 | 131.43 | | 0.68 | 7.73 |
| 6A_ | 0.00 | 4.80 | 7.83 | 15.07 | 0.00 | 98.10 | 266.28 | 10.43 | 92.89 | | 0.65 | 8.25 |
| 6D_ | 0.00 | 0.38 | 7.47 | 14.67 | 0.00 | 93.35 | 269.05 | 8.89 | 87.56 | 0.16 | 0.47 | |
| 7S_ | 37.71 | 0.00 | 11.38 | 22.05 | 0.00 | 108.65 | 245.55 | 13.66 | 73.67 | | 0.97 | |
| 7A_ | 38.05 | 5.36 | 10.73 | 21.45 | 0.00 | 106.42 | 240.59 | 12.31 | 36.44 | | 0.51 | |
| 7D_ | 477.25 | 0.20 | 0.00 | 20.62 | 0.00 | 99.84 | 225.82 | 12.09 | 106.97 | | 0.76 | |
| 8S_ | 0.00 | 0.00 | 5.33 | 11.36 | 0.00 | 58.84 | 404.28 | 10.39 | 73.30 | | 0.66 | |
| 8A_ | 0.00 | 0.00 | 4.49 | 10.02 | 0.00 | 57.60 | 459.79 | 8.06 | 28.26 | | 0.51 | |
| 8D_ | 0.00 | 0.00 | 5.45 | 10.75 | 0.00 | 60.31 | 400.81 | 8.84 | 51.55 | | 0.40 | |
| 9S_ | 26.79 | 4.29 | 42.61 | 32.18 | 0.00 | 464.72 | 346.32 | 14.44 | 71.12 | | 1.05 | 2.77 |
| 9A_ | 28.33 | 4.90 | 45.89 | 32.70 | 0.00 | 485.78 | 345.13 | 13.39 | 38.62 | | 0.80 | 2.85 |
| 9D_ | 28.09 | 5.23 | 44.38 | 31.63 | 0.00 | 450.80 | 324.28 | 13.24 | 251.90 | | 0.40 | |
| 10S_ | 4.49 | 0.00 | 4.19 | 11.67 | 0.00 | 148.60 | 192.56 | 13.24 | 194.67 | | 0.59 | |
| 10A_ | 0.00 | 3.24 | 4.25 | 10.94 | 0.00 | 136.19 | 199.84 | 6.97 | 19.57 | | 0.58 | |
| 10D_ | 0.00 | 0.00 | 4.38 | 11.35 | 1.24 | 142.53 | 185.98 | 7.72 | 143.24 | | 0.49 | 53.60 |
| 11S_ | 0.00 | 1.94 | 5.01 | 11.11 | 0.00 | 54.12 | 345.53 | 8.19 | 7.85 | | 0.38 | |
| 11A_ | 1.22 | 3.91 | 5.95 | 10.52 | 0.00 | 54.99 | 263.76 | 8.51 | 5.81 | | 0.30 | 0.98 |
| 11D_ | 0.00 | 3.53 | 5.81 | 11.14 | 0.00 | 58.34 | 264.61 | 8.49 | 4.54 | | 0.40 | 3.70 |
| 12S_ | 7.20 | 0.66 | 20.85 | 21.74 | 0.00 | 134.87 | 218.77 | 0.10 | 209.33 | 2.01 | 0.85 | |
| 12A_ | 6.81 | 0.00 | 18.28 | 19.20 | 0.00 | 124.90 | 192.99 | 8.02 | 158.39 | | 0.67 | 3.35 |
| 12D_ | 8.33 | 3.48 | 20.50 | 20.69 | 0.00 | 163.89 | 234.38 | 5.29 | 382.14 | | 0.65 | 3.57 |
| 13S_ | 0.00 | 0.00 | 4.28 | 11.35 | 0.00 | 82.00 | 235.34 | 9.79 | 121.78 | | 0.45 | 19.30 |
| 13A_ | 4.14 | 0.00 | 4.23 | 10.25 | 0.00 | 82.44 | 317.53 | 8.28 | 24.24 | | 0.35 | 17.81 |
| 13D_ | 0.00 | 1.14 | 4.25 | 10.54 | 0.00 | 78.33 | 228.82 | 7.95 | 91.79 | | 0.50 | |
| 14S_ | 21.46 | 5.10 | 14.51 | 13.86 | 0.00 | 679.85 | 723.18 | 43.67 | 4.51 | | 0.50 | |
| 14A_ | 20.40 | 6.01 | 12.26 | 13.76 | 0.00 | 647.41 | 657.56 | 37.39 | 12.91 | | 0.35 | |
| 14D_ | 22.80 | 4.12 | 15.40 | 14.47 | 0.00 | 709.57 | 715.39 | 46.76 | 69.87 | | 1.08 | |
| 15S_ | 0.00 | 5.50 | 13.65 | 16.43 | 0.00 | 343.78 | 240.18 | 12.66 | 100.91 | 3.28 | 0.57 | |
| 15A_ | 0.00 | 4.21 | 13.45 | 16.51 | 0.00 | 345.53 | 243.68 | 6.56 | 23.58 | | 0.59 | |
| 15D_ | 0.00 | 5.52 | 14.01 | 16.86 | 0.00 | 348.37 | 245.70 | 11.95 | 110.12 | 2.77 | 0.79 | |
| 16s_ | 0.00 | 1.30 | 5.34 | 16.49 | 2.90 | 93.74 | 136.78 | 15.90 | 139.92 | 0.39 | 0.67 | 115.11 |
| 16a_ | 0.00 | 0.59 | 4.13 | 16.62 | 3.53 | 97.63 | 127.17 | 12.81 | 291.99 | 0.09 | 0.15 | 115.11 |
| 16d_ | 0.00 | 2.36 | 5.20 | 15.28 | 3.04 | 90.83 | 127.21 | 11.98 | 244.43 | 0.33 | 0.28 | 109.87 |
| 17s_ | 0.00 | 0.96 | 5.75 | 19.57 | 7.22 | 187.72 | 220.25 | 10.53 | 15.32 | 0.21 | 0.81 | 349.27 |
| 17a_ | 6.24 | 1.85 | 6.01 | 17.92 | 7.54 | 207.28 | 163.55 | 11.13 | 33.16 | 0.28 | 0.68 | 352.61 |
| 17d_ | 0.00 | 0.00 | 5.71 | 18.06 | 11.14 | 201.03 | 183.81 | 10.00 | 33.10 | 0.11 | 0.98 | |
| 18s_ | 0.00 | 0.00 | 3.47 | 16.22 | 1.02 | 59.51 | 343.76 | 11.49 | 178.91 | 0.38 | 0.28 | |
| 18a_ | 0.00 | 2.69 | 3.60 | 15.64 | 0.89 | 57.81 | 356.16 | 8.13 | 53.19 | 0.26 | 0.16 | 56.49 |
| 18d_ | 0.00 | 3.21 | 3.30 | 15.81 | 1.75 | 56.75 | 251.35 | 12.64 | 107.71 | 0.84 | 0.18 | 54.69 |
| 19s_ | 11.46 | 0.00 | 4.14 | 17.14 | 19.91 | 95.85 | 138.09 | 12.86 | 217.87 | 0.31 | 0.82 | |
| 19d_ | 11.00 | 0.00 | 4.60 | 16.83 | 1.94 | 84.90 | 137.56 | 12.32 | 276.39 | 0.26 | 0.56 | |
| 20s_ | 8.42 | 2.51 | 7.02 | 23.45 | 8.29 | 320.54 | 212.78 | 14.00 | 7.77 | 0.08 | 0.37 | |
| 20a_ | 7.86 | 2.21 | 6.77 | 23.05 | 1.17 | 303.20 | 196.44 | 12.09 | 4.45 | 0.20 | 0.25 | 61.94 |
| 20d_ | 0.00 | 2.24 | 7.01 | 22.83 | 7.61 | 311.92 | 202.98 | 13.87 | 27.20 | 0.31 | 0.41 | 64.67 |
| 21s_ | 0.00 | 1.20 | 5.26 | 23.20 | 3.99 | 126.93 | 174.52 | 9.29 | 10.37 | 0.12 | 0.69 | 205.81 |
| 21a_ | 6.47 | 1.10 | 4.40 | 22.25 | 4.89 | 133.91 | 143.82 | 9.45 | 155.70 | 0.30 | 0.14 | 203.13 |
| 21d_ | 0.00 | 0.00 | 4.98 | 23.31 | 10.39 | 128.13 | 177.92 | 9.14 | 147.52 | 0.73 | 0.88 | |
| 22s_ | 0.00 | 0.00 | 5.30 | 18.51 | 2.50 | 91.84 | 196.52 | 5.60 | 364.68 | 0.10 | 1.07 | |
| 22a_ | 8.80 | 0.00 | 5.62 | 18.95 | 12.23 | 94.39 | 189.52 | 8.54 | 414.19 | 0.22 | 0.73 | |
| 22d_ | 0.00 | 3.28 | 4.97 | 18.12 | 1.40 | 88.45 | 177.46 | 9.72 | 37.06 | 0.30 | 0.98 | |
| 23S_ | 8.87 | 0.08 | 1.42 | 11.81 | 0 | 112.33 | 289.82 | 18.78 | 116.15 | | 0.41 | |
| 23A_ | 8.22 | 1.3 | 2.16 | 11.75 | 0 | 89 | 270.16 | 8.19 | 24.43 | | 0.25 | |
| 23D_ | 7.75 | 1.58 | 2.56 | 11.47 | 0 | 86.88 | 226.34 | 7.28 | 142.9 | | 0 | |
| 24A_ | 7.74 | 1.4 | 4.32 | 18.68 | 5.58 | 226.08 | 163.68 | 9.51 | 16.37 | | 0.27 | |
| 25S_ | 7.18 | 0.32 | 1.48 | 12.59 | 0.67 | 281.19 | 127.87 | 8.19 | 260.2 | | 0.45 | |
| 25A_ | 6.49 | 0.1 | 2.38 | 13.47 | 0 | 292 | 124.25 | 7.87 | 209.1 | | 0.08 | |
| 25D_ | 6.51 | 0 | 2.55 | 12.03 | 0 | 280.99 | 125.91 | 8.59 | 222.25 | | 0.2 | |
| 26S_ | 3.4 | 2.39 | 1.89 | 11.73 | 0 | 71.7 | 234.83 | 4.77 | 21.2 | | 0.31 | |
| 26A_ | 2.6 | 0.74 | 0.14 | 8.82 | 0 | 66.54 | 232.99 | 6.51 | 65.12 | | 0.21 | |
| 26D_ | 3.03 | 1.68 | 1.48 | 9.15 | 0 | 49.34 | 190.91 | 8.76 | 119.82 | | 0.27 | |
| CSF-16_ | 0 | 0 | 0 | 1.36 | 1.34 | 7.97 | 40.43 | 16.03 | 0 | 0.54 | 0.71 | |
| CSF-18_ | 0 | 0 | 0 | 1.74 | 0.64 | 7.88 | 40.27 | 23.73 | 0 | 0.54 | 0.75 | 1.23 |
| CSF-21_ | 0 | 0 | 0 | 3.37 | 3.14 | 11.85 | 55.44 | 21.39 | 0 | 0.6 | 0.5 | |
| CSF-22_ | 0.26 | 0 | 0 | 2.17 | 32.88 | 9.34 | 48.91 | 22.88 | 0 | 0.71 | 1.02 | |
| CSF-23_ | 0 | 0 | 0 | 0.91 | 1.74 | 3.57 | 17.68 | 15.94 | 0 | 0.53 | 0.58 | |
| CSF-25_ | 0 | 0 | 0 | 0.71 | 0.42 | 7.92 | 10.09 | 7.91 | 0 | 0.22 | 0.14 | |
| csf-1_ | 0 | 0 | 0 | 3.23 | 3.19 | 8.71 | 52.58 | 23.34 | 0 | 0.51 | 0.99 | |
| csf-2_ | 0 | 0 | 0 | 3.09 | 3.3 | 11.64 | 62.86 | 19.79 | 0 | 0.39 | 1.12 | |
| csf-5_ | 0 | 0 | 0 | 2.07 | 3.64 | 16.36 | 63.12 | 26.54 | 0 | 0.94 | 1.52 | |
| csf-6_ | 0 | 0 | 0 | 2.78 | 0 | 10.74 | 54 | 19.84 | 0 | 0.54 | 0.92 | |
| csf-7_ | 0 | 0 | 0 | 3.67 | 1.48 | 13.65 | 88.85 | 27.72 | 0 | 0.51 | 1.13 | |
| csf-9_ | 0 | 0 | 0 | 2.24 | 0.48 | 10.78 | 16.06 | 6.15 | 0 | 0.11 | 0.32 | |
| csf-10_ | 0 | 0 | 0 | 2.71 | 2.18 | 10.92 | 45.41 | 21.49 | 0 | 0.59 | 0.95 | |
| csf-11_ | 0 | 0 | 0 | 2.74 | 1.59 | 8.52 | 63.11 | 24.48 | 0 | 0.75 | 1.56 | |
| csf-14_ | 0 | 0 | 0 | 1.94 | 1.68 | 17.57 | 65.18 | 18.39 | 0 | 0.29 | 0.55 | |
| csf-15_ | 0 | 0 | 0 | 3.23 | 2.17 | 17.11 | 51.34 | 26.56 | 0 | 0.5 | 1.29 | |
| AVERAGE CSF | 0.11 | 0.00 | 0.22 | 2.77 | 2.54 | 13.08 | 59.14 | 24.46 | 0.00 | 0.52 | 0.92 | 1.23 |
| 5D_ | 0.00 | 509.22 | 4.99 | | | | 4.03 | | 0.00 | 0.01 | 18.13 | 1359.40 |
| 6S_ | 0.00 | 600.31 | 8.66 | 2.60 | | 18.18 | 72.31 | | 15.19 | 9.79 | 25.67 | 1266.83 |
| 6A_ | 4.21 | 634.27 | 8.50 | | | 11.54 | 45.41 | | 13.65 | 11.72 | 27.17 | 913.83 |
| 6D_ | 0.00 | 614.17 | 0.00 | | | 11.29 | 43.93 | 10.28 | 18.42 | 14.46 | 26.39 | 848.71 |
| 7S_ | 5.85 | 549.59 | 1.05 | | | | 12.83 | | 0.00 | 5.69 | 40.43 | 1282.96 |
| 7A_ | 1.04 | 502.32 | 3.87 | | | | 1.83 | | 0.88 | 2.24 | 39.66 | 1372.09 |
| 7D_ | 5.00 | 496.52 | 3.14 | | | | 5.24 | | 0.00 | 5.81 | 35.19 | 2193.10 |
| 8S_ | 2.73 | 506.88 | 1.53 | | | | 129.44 | | 6.77 | 0.01 | 21.96 | 1322.21 |
| 8A_ | 1.41 | 503.80 | 0.00 | 0.42 | | | 25.59 | | 0.00 | 0.01 | 19.65 | 863.20 |
| 8D_ | 0.78 | 505.77 | 5.76 | | | 16.50 | 57.59 | | 20.93 | 2.99 | 22.21 | 1100.08 |
| 9S_ | 5.10 | 541.67 | 19.20 | | | | 4.82 | | 38.73 | 0.01 | 60.06 | 761.82 |
| 9A_ | 2.56 | 545.27 | 20.83 | 1.67 | | | 3.89 | | 36.26 | 2.41 | 57.81 | 293.77 |
| 9D_ | 4.40 | 546.16 | 21.39 | 1.87 | | | 22.90 | | 37.42 | 0.01 | 54.17 | 2693.80 |
| 10S_ | 7.01 | 503.86 | 6.18 | | | | 3.48 | | 13.70 | 0.01 | 18.97 | 2782.91 |
| 10A_ | 0.00 | 499.94 | 0.00 | | | | 0.82 | | 8.99 | 0.01 | 17.30 | 773.16 |
| 10D_ | 6.61 | 509.66 | 5.24 | | | | 1.38 | | 8.67 | 0.01 | 18.03 | 987.05 |
| 11S_ | | 524.44 | 2.47 | | 0.04 | 4.28 | 34.86 | | 11.82 | 0.01 | 19.67 | 1268.36 |
| 11A_ | 5.08 | 508.50 | 0.61 | 1.06 | 0.04 | | 0.77 | | 4.63 | 5.84 | 17.71 | 475.93 |
| 11D_ | 2.00 | 503.79 | 7.81 | 0.88 | 0.04 | | 2.81 | | 4.58 | 3.81 | 18.01 | 504.33 |
| 12S_ | 3.10 | 525.77 | 11.91 | 1.57 | 0.04 | 51.02 | 165.83 | | 12.07 | 0.01 | 16.26 | 2312.62 |
| 12A_ | 1.20 | 507.19 | 0.00 | | 0.04 | 43.10 | 154.07 | | 0.00 | 0.01 | 13.63 | 1993.53 |
| 12D_ | 0.75 | 528.27 | 0.00 | 0.69 | 0.04 | 8.12 | 31.13 | | 0.00 | 0.01 | 17.93 | 904.48 |
| 13S_ | 4.63 | 503.39 | 5.96 | 1.21 | 0.04 | 7.39 | 15.64 | | 9.83 | 0.01 | 18.72 | 2252.13 |
| 13A_ | 3.36 | 487.10 | 5.67 | 0.90 | 0.04 | | 2.72 | | 0.00 | 0.01 | 17.72 | 830.76 |
| 13D_ | 8.01 | 524.70 | 7.64 | | 0.04 | 7.01 | 18.29 | | 0.00 | 0.01 | 15.31 | 1813.65 |
| 14S_ | 6.69 | 733.05 | 6.63 | 10.39 | 0.04 | | 2.43 | | 54.46 | 0.01 | 182.78 | 153.44 |
| 14A_ | 6.37 | 602.61 | 6.06 | 8.71 | 0.04 | 5.52 | 31.01 | | 0.00 | 0.01 | 158.28 | 1549.15 |
| 14D_ | 4.41 | 704.68 | 6.54 | 9.60 | 0.04 | | 82.36 | | 50.00 | 0.01 | 180.17 | 637.23 |
| 15S_ | 2.37 | 551.72 | 3.68 | 1.65 | | 53.41 | 134.40 | | 0.00 | 0.01 | 16.73 | 2121.52 |
| 15A_ | 5.00 | 526.04 | 3.22 | 1.36 | | | 7.07 | | 23.49 | 0.01 | 15.60 | 1391.49 |
| 15D_ | 5.77 | 521.38 | 3.74 | 1.60 | | 55.71 | 134.85 | | 0.00 | 0.01 | 18.77 | 2157.73 |
| 16s_ | 7.96 | 514.39 | 0.00 | | 4.79 | | 22.09 | 1.01 | 39.80 | 0.01 | 12.66 | 841.55 |
| 16a_ | 6.53 | 473.18 | 0.00 | | 3.85 | 10.65 | 24.02 | 20.05 | 0.00 | 0.01 | 12.02 | 2752.61 |
| 16d_ | 146.33 | 485.28 | 0.00 | 1.62 | 3.15 | 18.40 | 40.20 | 43.19 | 0.00 | 0.01 | 11.36 | 1840.07 |
| 17s_ | 9.14 | 528.72 | 0.00 | | 0.27 | | 15.18 | 24.62 | 4.02 | 0.01 | 22.33 | 354.80 |
| 17a_ | 3.52 | 544.15 | 0.00 | 1.94 | 0.22 | | 42.45 | 59.95 | 0.00 | 0.01 | 23.38 | 359.00 |
| 17d_ | 5.79 | 537.31 | 0.00 | 1.98 | 2.01 | | 7.46 | 20.83 | 21.03 | 0.01 | 23.75 | 888.88 |
| 18s_ | 7.29 | 462.02 | 0.00 | 4.68 | | 48.04 | 204.72 | 33.04 | 0.00 | 6.49 | 23.45 | 3481.50 |
| 18a_ | 8.89 | 465.53 | 0.00 | 5.20 | | 5.66 | 23.74 | 25.60 | 0.00 | 6.15 | 23.62 | 1349.99 |
| 18d_ | 8.67 | 454.16 | 0.00 | 4.88 | 0.23 | 12.00 | 55.00 | 16.38 | 0.00 | 6.54 | 23.52 | 2961.81 |
| 19s_ | 7.25 | 482.70 | 0.00 | | 1.79 | 46.82 | 133.97 | 12.50 | 0.00 | 0.01 | 18.58 | 1477.81 |
| 19d_ | 1.67 | 506.31 | 0.00 | | 0.24 | 56.98 | 154.87 | 27.50 | 0.00 | 0.01 | 17.09 | 1965.27 |
| 20s_ | 1.39 | 549.53 | 0.00 | | 0.58 | 2.04 | 28.30 | 26.17 | 21.33 | 7.52 | 42.73 | 220.67 |
| 20a_ | 7.74 | 551.05 | 0.77 | | 0.46 | | 11.67 | 77.54 | 17.76 | 7.04 | 38.99 | 277.74 |
| 20d_ | 8.88 | 554.61 | 0.00 | 3.35 | 0.84 | 6.44 | 28.37 | 35.12 | 0.00 | 6.73 | 41.47 | 844.63 |
| 21s_ | 0.00 | 469.87 | 0.00 | 2.35 | | | 6.42 | 91.30 | 0.00 | 0.01 | 13.55 | 224.42 |
| 21a_ | 7.82 | 477.21 | 9.02 | | 0.18 | | 24.43 | 81.66 | 16.30 | 0.01 | 13.24 | 2165.61 |
| 21d_ | 0.00 | 476.92 | 0.00 | 1.82 | | 32.29 | 86.51 | 6.18 | 0.00 | 0.01 | 13.28 | 1726.73 |
| 22s_ | 7.44 | 475.60 | 0.00 | | 0.31 | 35.84 | 50.25 | 38.52 | 15.02 | 3.08 | 15.78 | 2918.89 |
| 22a_ | 6.57 | 425.60 | 0.00 | 2.04 | 0.14 | 102.51 | 282.95 | 46.31 | 11.26 | 0.01 | 14.56 | 2050.85 |
| 22d_ | 6.29 | 443.76 | 0.00 | | 1.76 | | 9.09 | 3.21 | 7.91 | 0.01 | 13.91 | 800.74 |
| 23S_ | 9.56 | | 0.2 | 0.28 | | 9.4 | 23.8 | 17.69 | 36.52 | 0.01 | 27.86 | 5377.09 |
| 23A_ | 6.11 | | 0.69 | 1.78 | | 2.5 | 5.2 | 61.89 | 0 | 2.04 | 27.36 | 1956.35 |
| 23D_ | 1.67 | | 0.72 | 1.86 | | 6.21 | 0.26 | 12.3 | 0.64 | 1.41 | 27.16 | 2190.95 |
| 24A_ | 7.76 | | 0.1 | 1.68 | | 1.62 | 2.43 | 4.12 | 6.01 | 0.01 | 24.62 | 983.33 |
| 25S_ | 3.66 | | 1.4 | 0.8 | | 36.11 | 79.63 | | 0 | 0.01 | 33.99 | 2271.84 |
| 25A_ | 9.48 | | 1.82 | 0.66 | | 6.22 | 13.09 | | 0 | 0.29 | 33.02 | 2692.98 |
| 25D_ | 4.27 | | 0.19 | 0.55 | | 15.94 | 25.8 | | 0 | 0.15 | 33.33 | 2583.34 |
| 26S_ | 8.77 | | 0.94 | 6.32 | | 4.5 | 2.57 | 42.31 | 7.27 | 0.79 | 21.36 | 1394.03 |
| 26A_ | 4.83 | | 0.18 | 2.34 | | 13.37 | 3.65 | 10.2 | 16.02 | 0.16 | 19.63 | 5100.61 |
| 26D_ | 6.95 | | 0.57 | 4.92 | | 31.57 | 24.27 | | 0 | 2.35 | 19.57 | 3681.37 |
| CSF-16_ | 1159.29 | 56.32 | 1.06 | | 0.34 | | 10.4 | 24.16 | 5.42 | 0 | 25.37 | 390.21 |
| CSF-18_ | 4.29 | 60.02 | 6.84 | | | | 18.09 | 11.22 | 13.32 | 0 | 50.45 | 692.3 |
| CSF-21_ | 0.73 | 85.15 | 7.56 | | | | 11.51 | 4.71 | 3.54 | 0 | 44.48 | 706 |
| CSF-22_ | 0.56 | 100.96 | 7.5 | 0.49 | | | 13.44 | 36.25 | 0 | 0 | 29.39 | 735.11 |
| CSF-23_ | 0.18 | 125.9 | 5.17 | 0.44 | | | 10.4 | 4.69 | 1.55 | 0 | 16.13 | 337.79 |
| CSF-25_ | 3272.11 | 21.23 | 0.83 | | | | 3.96 | 3.3 | 0 | 0 | 16.92 | 301.33 |
| csf-1_ | 0 | 77.04 | 7.62 | | | | 8.97 | 41.82 | 1.1 | 0 | 41.93 | 555.55 |
| csf-2_ | 0 | 106.87 | 0.85 | | | | 8.84 | 33.81 | 3.9 | 0 | 32.32 | 387.39 |
| csf-5_ | 0 | 124.92 | 6.42 | | | | 13.29 | 56.92 | 6.07 | 0 | 36.46 | 785.17 |
| csf-6_ | 4875.53 | 155.71 | 8.23 | | | | 9.4 | 78.69 | 3.33 | 0 | 26.16 | 247.43 |
| csf-7_ | 0 | 139.5 | 8.22 | | | | 5.64 | 48.66 | 12.53 | 0 | 49.02 | 425.19 |
| csf-9_ | 0 | 179.88 | 5.23 | | | | 3.5 | 38.32 | 8.83 | 0 | 10.28 | 192.63 |
| csf-10_ | 0 | 87.61 | 8.44 | | | | 8.42 | 40.48 | 18.63 | 0 | 29.65 | 521.64 |
| csf-11_ | 4516.83 | 76.73 | 8.53 | | | | 12.66 | 73.21 | 3.89 | 0 | 34.66 | 723.23 |
| csf-14_ | 12.74 | 119.09 | 6.92 | | | | 13.57 | 37.45 | 7.23 | 0 | 31.71 | 666.64 |
| csf-15_ | 0 | 111.58 | 8.15 | | | | 14.71 | 25.94 | 10.16 | 0 | 40.34 | 503.43 |
| AVERAGE CSF | 1160.23 | 101.78 | 10.52 | 0.47 | 0.05 | 0.00 | 9.85 | 34.97 | 3.83 | 0.00 | 40.23 | 445.04 |
| 5D_ | 227.48 | | 0.00 | 2996.28 | 8.71 | 17.39 | 3203.5 | 2.92 | 4985.8 | | | |
| 6S_ | 409.20 | 4.23 | 0.88 | 54.67 | 4.5 | 13.77 | 6049.7 | 0.11 | 6261.5 | | | |
| 6A_ | 415.93 | | 1.08 | 2917.27 | 4.3 | 15.27 | 6202.9 | 0.08 | 6360.6 | | | |
| 6D_ | 408.31 | | 0.00 | 3059.60 | 4.78 | 16.09 | 5995.4 | 0.18 | 6271.4 | | | |
| 7S_ | 647.83 | | 1.02 | 3876.03 | 6.69 | 15.00 | 8972.8 | 1.72 | 9389.5 | | | |
| 7A_ | 621.55 | | 0.00 | 4126.58 | 5.91 | 25.27 | 8947.4 | 1.91 | 9757.5 | | | |
| 7D_ | 587.28 | 5.47 | 0.00 | 4583.16 | 5.76 | 252.72 | 8089.6 | 84.46 | 9744.6 | | | |
| 8S_ | 283.41 | | 0.00 | 4203.36 | 9.28 | 8.35 | 6787.9 | 1.02 | 7691.7 | | | |
| 8A_ | 282.89 | | 0.00 | 4219.57 | 9.52 | 10.49 | 6459.5 | 2.10 | 7348.3 | 1.97 | | |
| 8D_ | 284.61 | | 0.00 | 3687.10 | 8.88 | 2.16 | 6648.6 | 2.36 | 7149.5 | | | |
| 9S_ | 538.30 | 3.95 | 0.00 | 2967.78 | 12.47 | 10.14 | 5693.1 | 0.34 | 8574.8 | | | |
| 9A_ | 535.56 | | 0.00 | 3127.73 | 12.17 | 10.13 | 5656.1 | 3.96 | 8906.5 | | 0.44 | |
| 9D_ | 502.78 | | 0.00 | 3570.99 | 10.47 | 10.04 | 5452.0 | 2.55 | 8673.8 | | | |
| 10S_ | 286.32 | | 1.08 | 3861.01 | 7.24 | 16.03 | 7989.0 | 1.86 | 7608.0 | 13.87 | | |
| 10A_ | 281.76 | 3.16 | 0.00 | 4143.07 | 7.69 | 81.65 | 7582.1 | 0.99 | 8145.2 | 4.16 | | |
| 10D_ | 276.56 | | 0.00 | 3259.61 | 5.64 | 264.29 | 7859.0 | 1.88 | 6804.9 | | | |
| 11S_ | 368.76 | 2.16 | 0.00 | 3993.42 | 7.7 | 8.40 | 6161.8 | 5.56 | 6484.3 | 16.20 | | |
| 11A_ | 354.28 | | 0.00 | 3481.45 | 6.5 | 0.82 | 5780.3 | 4.21 | 6153.8 | 7.02 | | |
| 11D_ | 356.32 | | 0.00 | 3936.29 | 5.37 | 10.94 | 5873.4 | 4.26 | 6162.5 | 7.20 | | 0.20 |
| 12S_ | 400.74 | | 0.00 | 4248.91 | 10.28 | 11.48 | 4302.9 | 0.40 | 6826.5 | | 0.58 | |
| 12A_ | 350.08 | | 0.00 | 3436.64 | 6.81 | 13.22 | 3927.2 | 0.62 | 5761.8 | | | |
| 12D_ | 383.32 | 2.97 | 0.00 | 3056.68 | 7.86 | 14.26 | 4288.4 | 1.35 | 5872.0 | 3.54 | | |
| 13S_ | 308.32 | | 0.00 | 4070.02 | 6.64 | 24.07 | 7629.5 | 2.47 | 7923.1 | 33.97 | | |
| 13A_ | 312.26 | | 0.00 | 4439.71 | 7.95 | 26.06 | 7255.5 | 1.91 | 8011.1 | 8.80 | | |
| 13D_ | 292.19 | 2.65 | 0.00 | 5280.03 | 6.08 | 30.46 | 7669.4 | 1.76 | 9214.2 | 21.61 | | |
| 14S_ | 467.22 | 0.20 | 0.00 | 2002.31 | 14.19 | 140.91 | 4606.4 | 13.53 | 5421.5 | | | |
| 14A_ | 421.20 | 0.20 | 0.00 | 2295.36 | 12.26 | 13.79 | 4222.0 | 5.13 | 5790.8 | | | |
| 14D_ | 455.82 | 0.20 | 0.00 | 1987.33 | 12.92 | 0.79 | 4739.3 | 6.14 | 5649.8 | | | |
| 15S_ | 358.29 | 0.20 | 0.00 | 3567.97 | 9.72 | 0.18 | 5818.5 | 5.33 | 6841.8 | 12.15 | | |
| 15A_ | 357.18 | 0.20 | 0.00 | 3790.72 | 9.04 | 15.17 | 5989.0 | 4.62 | 6819.7 | 15.42 | | |
| 15D_ | 376.23 | 0.20 | 0.00 | 3727.91 | 11.29 | 0.26 | 5729.5 | 10.68 | 6806.2 | 10.22 | | |
| 16s_ | 357.45 | 0.20 | 0.00 | 3186.51 | 0.41 | | 6449.7 | 143.17 | 5406.2 | | | |
| 16a_ | 337.03 | 5.17 | 0.00 | 3473.77 | 0.35 | | 6488.4 | 146.65 | 5652.3 | | | |
| 16d_ | 332.56 | | 0.00 | 2995.10 | 0.42 | | 5908.7 | 132.75 | 4902.8 | | | |
| 17s_ | 371.21 | 13.20 | 0.00 | 3950.30 | 15.50 | | 5978.0 | 14.08 | 7176.6 | | | |
| 17a_ | 399.67 | | 2.54 | 3204.47 | 13.46 | | 5837.0 | | 6409.8 | | | |
| 17d_ | 406.06 | | 0.00 | 3364.80 | 16.16 | | 5668.2 | | 6401.8 | | | |
| 18s_ | 327.63 | | 0.00 | 5224.63 | 21.56 | | 8264.2 | 0.14 | 9670.8 | | 0.15 | |
| 18a_ | 343.10 | | 0.00 | 5139.94 | 21.02 | | 8271.1 | 122.56 | 9772.3 | | | |
| 18d_ | 312.48 | 3.04 | 0.00 | 4679.31 | 18.96 | | 7841.0 | 120.34 | 9162.5 | | | |
| 19s_ | 399.20 | 12.93 | 0.00 | 2893.08 | 14.02 | | 6963.0 | 205.80 | 5061.5 | | | |
| 19d_ | 409.99 | 0.20 | 0.00 | 3675.06 | 11.62 | | 7397.1 | 11.20 | 5772.9 | | | |
| 20s_ | 429.38 | 0.20 | 0.00 | 3993.29 | 16.13 | | 6318.5 | 17.61 | 7909.5 | | | |
| 20a_ | 392.02 | 0.20 | 0.00 | 3876.67 | 15.15 | | 6013.9 | | 7708.3 | | | |
| 20d_ | 419.14 | 0.20 | 0.00 | 3715.11 | 15.36 | | 5975.7 | 17.73 | 7380.9 | | | |
| 21s_ | 424.15 | 0.20 | 0.00 | 3433.48 | 12.78 | | 5237.0 | | 5292.9 | | | |
| 21a_ | 388.94 | | 0.00 | 4056.14 | 10.80 | | 5104.6 | 134.14 | 5790.2 | | | |
| 21d_ | 413.66 | | 0.00 | 3890.39 | 13.49 | | 5168.2 | 1.28 | 5684.3 | | | |
| 22s_ | 413.14 | | 0.00 | 4619.25 | 22.20 | | 7693.1 | | 8451.7 | | | |
| 22a_ | 380.69 | 14.87 | 0.00 | 4525.19 | 16.22 | | 7968.8 | 0.87 | 8721.8 | | | |
| 22d_ | 359.23 | 7.03 | 0.00 | 4160.92 | 14.69 | | 7579.8 | 10.96 | 8120.6 | | | |
| 23S_ | 188.25 | | 0.46 | 8247.38 | 6.84 | | 7235.8 | 0.37 | 8213.5 | | 2.05 | |
| 23A_ | 184.51 | | 1.02 | 6617.89 | 7.13 | | 6889.0 | 0.4 | 7240.6 | | 0.24 | |
| 23D_ | 172.02 | | 0.57 | 6161.9 | 7.15 | | 6728.9 | 8.5 | 7308.9 | | 0.29 | |
| 24A_ | 207.99 | | 0.41 | 5175.95 | 5.84 | | 5511.0 | 0.11 | 5906.5 | | 0.21 | |
| 25S_ | 193.93 | | 3.29 | 4923.17 | 8.26 | | 5218.5 | 3.56 | 5941.5 | | 1.23 | |
| 25A_ | 189.31 | | 0 | 4788.37 | 6.02 | | 5267.6 | 3.72 | 5942.8 | | 0.17 | |
| 25D_ | 187.48 | | 0.18 | 4752.8 | 6.22 | | 5033.5 | 3.32 | 5678.2 | | 1.27 | |
| 26S_ | 168.05 | | 0.36 | 7665.38 | 5.66 | | 8180.9 | 7.28 | 9240.0 | | 0.36 | |
| 26A_ | 153.09 | | 0 | 7244.29 | 5.39 | | 7398.3 | 6.51 | 8383.4 | | 0.18 | |
| 26D_ | 148.36 | | 0.54 | 6973.83 | 5.57 | | 7405.8 | 0.21 | 8190.8 | | 1.41 | |
| CSF-16_ | 4.76 | 0.02 | 0 | 382.87 | 7.12 | | 208.25 | | 685.05 | | | |
| CSF-18_ | 17.56 | 0.02 | 0 | 539.11 | 7.73 | | 398.59 | | 1546.73 | | | |
| CSF-21_ | 7.95 | 0.02 | 0 | 633.66 | 9.37 | | 302.67 | | 1108.23 | | | |
| CSF-22_ | 8.04 | 0.02 | 0 | 816.07 | 10.99 | | 378 | 0.68 | 1336.56 | | | |
| CSF-23_ | 4.47 | 0.02 | 0 | 353.96 | 6.79 | | 220.1 | 1.4 | 650.39 | | | |
| CSF-25_ | 1.99 | 0.02 | 0 | 187.3 | 3.47 | | 93.12 | | 315.01 | | | |
| csf-1_ | 7.42 | 0.45 | 0 | 373.06 | 10.02 | | 280.48 | | 1236.82 | | | |
| csf-2_ | 9.89 | 0.54 | 0 | 1112.38 | 10.03 | | 564.64 | | 2487.9 | | | |
| csf-5_ | 8.46 | 0.6 | 0 | 646.66 | 18.26 | | 489.14 | | 1467.18 | | | |
| csf-6_ | 8.64 | 0.84 | 0 | 358.74 | 8.45 | | 281.67 | | 1095.88 | | | |
| csf-7_ | 12.67 | 0.71 | 0 | 364.69 | 12.66 | | 389.5 | | 1558.31 | | | |
| csf-9_ | 5.9 | 0.02 | 0 | 291.79 | 7.06 | | 149.78 | | 730.55 | | | |
| csf-10_ | 6.78 | 0.35 | 0 | 838.79 | 12.82 | | 467.76 | | 1775.08 | | | |
| csf-11_ | 6.5 | 0.69 | 0 | 829.07 | 12.21 | | 407.23 | | 1614.87 | | | |
| csf-14_ | 4.91 | 0.02 | 0 | 458.4 | 12.1 | | 221.15 | | 1064.97 | | | |
| csf-15_ | 8.66 | 0.02 | 0 | 886.67 | 16.24 | | 326.36 | | 1372.45 | | | |
| AVERAGE CSF | 9.06 | 0.27 | 0.10 | 556.46 | 13.04 | | 384.17 | 1.04 | 1439.30 | | | |

| | | | | | | | | | | | | |
|---|---|---|---|---|---|---|---|---|---|---|---|---|
| SDEV CSF | 0.25603 | 0 | 0.2958 | 1.2862 | 6.28068 | 7.449573 | 27.3399 | 11.047 | 0 | 0.2011 | 0.3561 | |
| COUNT CSF | 26 | 26 | 26 | 26 | 26 | 26 | 26 | 26 | 26 | 16 | 26 | 1 |
| control_csf_ | 0 | 0 | 0.08 | 2.42 | 1.54 | 7.59 | 60.47 | 24.83 | 0 | 0.65 | 0.87 | |
| ds3_ | | | | 20 | 200 | 200 | 100 | 25 | | 20 | 20 | |
| ds3_ | | | | 20.12 | 204.66 | 202.89 | 101.42 | 24.8 | | 20.28 | 19.86 | |
| SET 2 PLAS | | | | | | | | | | | | |
| AVERAGE | 6.98 | 1.21 | 4.18 | 15.77 | 2.93 | 235.36 | 236.40 | 8.64 | 40.11 | | 0.26 | 6.98 |
| SDEVIATION | 17.8191 | 0.8481 | 1.9086 | 4.1935 | 1.88599 | 583.0247 | 128.557 | 4.2878 | 57.1203 | | 0.1408 | |
| COUNT | 53 | 53 | 53 | 53 | 53 | 53 | 53 | 53 | 53 | 0 | 53 | 1 |
| SET 1 PLASMAS | | | | | | | | | | | | |
| AVERAGE | 13.95 | 2.34 | 9.39 | 17.71 | 1.76 | 183.52 | 269.48 | 11.93 | 103.36 | 0.57 | 0.59 | 90.40 |
| SDEVIATION | 59.2994 | 2.1797 | 8.8082 | 5.5591 | 3.75282 | 169.4957 | 124.468 | 7.4093 | 98.9305 | 0.8368 | 0.26 | 88.8733 |
| COUNT | 65 | 65 | 65 | 65 | 65 | 65 | 65 | 65 | 65 | 25 | 65 | 33 |

| | | | | | | | | | | | | |
|---|---|---|---|---|---|---|---|---|---|---|---|---|
| SDEV CSF | 2556.8591 | 39.9884 | 6.2172 | 0.0354 | 0.0965 | 0 | 3.69301 | 22.7201 | 5.13024 | 0 | 22.9673 | 216.4373 |
| COUNT CSF | 26 | 16 | 26 | 2 | 11 | 10 | 26 | 16 | 26 | 26 | 26 | 26 |
| control_csf_ | 13776.83 | 35.72 | 7.34 | 0.04 | | | 8.08 | 26.33 | 2.38 | 1.03 | 52.36 | 559.92 |
| ds3_ | 10000 | 200 | 20 | | | | 200 | 500 | 500 | | 100 | 100 |
| ds3_ | 9693.34 | 229.65 | 18.99 | | | | 208.31 | 552.01 | 782.65 | | 103.88 | 147.15 |
| SET 2 PLAS | | | | | | | | | | | | |
| AVERAGE | 0.02 | | 4.66 | 0.15 | 0.25 | 0.58 | 31.86 | 0.32 | 80.89 | 0.18 | 29.75 | 1044.16 |
| SDEVIATION | 0.1703271 | | 2.4094 | | 1.4193 | 0.89253 | 27.8563 | | 59.9765 | 0.5316 | 34.7899 | 607.9246 |
| COUNT | 53 | 0 | 53 | 1 | 47 | 53 | 53 | 1 | 53 | 53 | 53 | 53 |
| SET 1 PLASMAS | | | | | | | | | | | | |
| AVERAGE | 6.98 | 528.30 | 4.44 | 2.91 | 0.76 | 26.83 | 41.50 | 33.85 | 11.20 | 2.05 | 33.48 | 1402.40 |
| SDEVIATION | 17.920319 | 61.8415 | 5.5487 | 2.5885 | 1.269 | 24.7982 | 56.7307 | 24.4985 | 13.3283 | 3.3312 | 35.6077 | 863.6599 |
| COUNT | 64 | 65 | 65 | 30 | 28 | 25 | 65 | 23 | 65 | 65 | 65 | 65 |

| | | | | | | | | | | | | |
|---|---|---|---|---|---|---|---|---|---|---|---|---|
| SDEV CSF | 3.497411 | 0.3139 | 0.1688 | 218.5154 | 5.0547 | | 165.73759 | 0.50912 | 604.24452 | | | |
| COUNT CSF | 26 | 16 | 26 | 26 | 26 | | 26 | 2 | 26 | | | |
| control_csf_ | 5.43 | 0.38 | 0 | 503.84 | 6.97 | | 230.02 | | 1021.9 | | | |
| ds3_ | 20 | 20 | | 250 | 20 | | 200 | | 200 | | | |
| ds3_ | 22.05 | 18.7 | | 320.97 | 20.73 | | 206.43 | | 208.88 | | | |
| SET 2 PLAS | | | | | | | | | | | | |
| AVERAGE | 332.95 | 1.51 | 3.95 | 3376.12 | 10.41 | | 7521.47 | 43.29 | 7723.44 | 0.97 | 1.15 | |
| SDEVIATION | 139.264 | | | 796.5626 | 4.9056 | | 1572.6452 | | 2088.8589 | | | |
| COUNT | 53 | | | 53 | 53 | 0 | 53 | 1 | 53 | 1 | 1 | |
| SET 1 PLASMAS | | | | | | | | | | | | |
| AVERAGE | 417.64 | 3.47 | 0.17 | 3705.25 | 9.61 | 31.32 | 6662.78 | 21.48 | 7452.05 | 11.33 | 0.45 | 0.20 |
| SDEVIATION | 131.1489 | | | 900.2746 | 4.7997 | 54.7434 | 1715.7929 | 47.0556 | 1705.8453 | 8.7178 | 0.2148 | |
| COUNT | 65 | | | 65 | 65 | 45 | 64 | 60 | 65 | 14 | 5 | |

t-Test: Two-Sample Assuming Equal Variances

| | Variable | ariable 2 |
|---|---|---|
| Mean | 332.95 | 417.64 |
| Variance | 19394 | 17200 |
| Observatio | 53 | 65 |
| Pooled Var | 18184 | |
| Hypothesi | 0 | |
| df | 116 | |
| t Stat | −3.393 | |
| P(T < = t) on | 0.0005 | |
| t Critical o | 1.6581 | |
| P(T < = t) tw | 0.0009 | |
| t Critical t | 1.9806 | |

| | 2HPAC | 3OHAN | 3OHKY | 3OMD | 4HBAC | 4HPAC | 4HPLA | 5HIAA | 5HT | 5HTP | ASC | DA |
|---|---|---|---|---|---|---|---|---|---|---|---|---|
| 23S_ | 8.87 | 0.08 | 1.42 | 11.81 | | 112.33 | 289.82 | 18.78 | 116.15 | 0.41 | 9.56 | 0.2 |
| 23A_ | 8.22 | 1.3 | 2.16 | 11.75 | | 89 | 270.16 | 8.19 | 24.43 | 0.25 | 6.11 | 0.69 |
| 23D_ | 7.75 | 1.58 | 2.56 | 11.47 | | 86.88 | 226.34 | 7.28 | 142.9 | | 1.67 | 0.72 |
| 24A_ | 7.74 | 1.4 | 4.32 | 18.68 | 5.58 | 226.08 | 163.68 | 9.51 | 16.37 | 0.27 | 7.76 | 0.1 |
| 25S_ | 7.18 | 0.32 | 1.48 | 12.59 | 0.67 | 281.19 | 127.87 | 8.19 | 260.2 | 0.45 | 3.66 | 1.4 |
| 25A_ | 6.49 | 0.1 | 2.38 | 13.47 | | 292 | 124.25 | 7.87 | 209.1 | 0.08 | 9.48 | 1.82 |
| 25D_ | 6.51 | | 2.55 | 12.03 | | 280.99 | 125.91 | 8.59 | 222.25 | 0.2 | 4.27 | 0.19 |
| 26S_ | 3.4 | 2.39 | 1.89 | 11.73 | | 71.7 | 234.83 | 4.77 | 21.2 | 0.3 | 8.77 | 0.94 |
| 26A_ | 2.6 | 0.74 | 0.14 | 8.82 | | 66.54 | 232.99 | 6.51 | 65.12 | 0.21 | 4.83 | 0.18 |
| 26D_ | 3.03 | 1.68 | 1.48 | 9.15 | | 49.34 | 190.91 | 8.76 | 119.82 | 0.27 | 6.95 | 0.57 |

15167779
15610000
2835.631
2899.87

| | DOPAC | G | GR | GSH | GSSG | HGA | HVA | HX | KYN | MEL | MET | MHPG |
|---|---|---|---|---|---|---|---|---|---|---|---|---|
| 23S_ | 0.28 | 9.4 | 23.8 | 17.69 | 36.52 | | 27.86 | 5377.09 | 188.25 | 0.46 | 8247.38 | 6.84 |
| 23A_ | 1.78 | 2.5 | 5.2 | 61.89 | | 2.04 | 27.36 | 1956.35 | 184.51 | 1.02 | 6617.89 | 7.13 |
| 23D_ | 1.86 | 6.21 | 0.26 | 12.3 | 0.64 | 1.41 | 27.16 | 2190.95 | 172.02 | 0.57 | 6161.9 | 7.15 |
| 24A_ | 1.68 | 1.62 | 2.43 | 4.12 | 6.01 | | 24.62 | 983.33 | 207.99 | 0.41 | 5175.95 | 5.84 |
| 25S_ | 0.8 | 36.11 | 79.63 | | | | 33.99 | 2271.84 | 193.93 | 3.29 | 4923.17 | 8.26 |
| 25A_ | 0.66 | 6.22 | 13.09 | | | 0.29 | 33.02 | 2692.98 | 189.31 | | 4788.37 | 6.02 |
| 25D_ | 0.55 | 15.94 | 25.8 | | | 0.15 | 33.33 | 2583.34 | 187.48 | 0.18 | 4752.8 | 6.22 |
| 26S_ | 6.32 | 4.5 | 2.57 | 42.31 | 7.27 | 0.79 | 21.36 | 1394.03 | 168.05 | 0.36 | 7665.38 | 5.66 |
| 26A_ | 2.34 | 13.37 | 3.65 | 10.2 | 16.02 | 0.16 | 19.63 | 5100.61 | 153.09 | | 7244.29 | 5.39 |
| 26D_ | 4.92 | 31.57 | 24.27 | | | 2.35 | 19.57 | 3681.37 | 148.36 | 0.54 | 6973.83 | 5.57 |

| | NA5HT | TRP | TRYPT | TYR | URIC | VMA1 | VMA2 | XAN | XANTHOSINE |
|---|---|---|---|---|---|---|---|---|---|
| 23S_ | 2.05 | 0.63 | 0.37 | 8681.66 | 27305.92 | 4.58 | 5.46 | 624.78 | 220.6 |
| 23A_ | 0.24 | 0.95 | 0.4 | 7779.54 | 26775.92 | 4.54 | 3.25 | 358.94 | 189.25 |
| 23D_ | 0.29 | 0.44 | 8.5 | 7712.2 | 26704.58 | 4.72 | 5.18 | 443.55 | 115.32 |
| 24A_ | 0.21 | | 0.11 | 6483.23 | 24094.22 | 6.55 | 6.96 | 92.84 | 72.71 |
| 25S_ | 1.23 | 2.46 | 3.56 | 6425.23 | 24982.39 | 5.49 | 7.33 | 222.27 | 35.56 |
| 25A_ | 0.17 | | 3.72 | 6363.69 | 24974.36 | 4.93 | 6.19 | 266.87 | 46.42 |
| 25D_ | 1.27 | 0.86 | 3.32 | 6062.19 | 24536.95 | 4.54 | 6.55 | 239.96 | 67.31 |
| 26S_ | 0.36 | 1.49 | 7.28 | 8588.34 | 23073.97 | 2.84 | 8.38 | 247.77 | 60.42 |
| 26A_ | 0.18 | | 6.51 | 7950.35 | 19934.82 | 2.57 | 4.79 | 343.64 | 64.71 |
| 26D_ | 1.41 | 1.22 | 0.21 | 7747.55 | 22153.09 | 3.01 | 5.9 | 292.89 | 39.27 |

TABLE AL-BB

| NM5HT | URIC | VMA1 | VMA2 | XAN | XANTHOSINE | 84* | 86* | 96* |
|---|---|---|---|---|---|---|---|---|
| 0 | 3166.05 | 0.00 | 0.00 | 272.60 | 12.56 | 0.14 | 7.69 | 0.95 |
| 0 | 2550.12 | 0.00 | 0.00 | 240.74 | 8.10 | 0.06 | 3.3 | 0.25 |
| 0 | 2752.43 | 0.00 | 0.00 | 244.56 | 11.93 | 0.16 | 5.36 | 1.16 |
| 0 | 5936.43 | 0.00 | 0.00 | 275.30 | 11.36 | 0.3 | 7.43 | 3.33 |
| 0 | 2823.94 | 0.00 | 0.00 | 297.33 | 12.12 | 0.25 | 4.86 | 3.68 |
| 0 | 6488.16 | 0.00 | 0.00 | 346.38 | 16.22 | 0.34 | 34.49 | 28.45 |
| 0 | 3134.53 | 0.00 | 0.00 | 401.21 | 13.44 | 0.12 | 50.35 | 24.3 |
| 0 | 3801.53 | 0.00 | 0.00 | 274.79 | 14.54 | 0.12 | 6.26 | 4.57 |
| 0 | 4731.90 | 0.00 | 0.00 | 220.33 | 9.42 | 0.2 | 9.18 | 4.27 |
| 0 | 8074.39 | 0.00 | 0.00 | 362.37 | 6.75 | 0.22 | 174.92 | 177.22 |
| 0.48 | 21905.3 | 7.02 | 6.9 | 585.16 | | 0.36 | 37.5 | 23.26 |
| 0 | 20552.7 | 6.17 | 5.85 | 490.53 | 10.75 | 4.76 | 36.74 | 61.15 |
| 0 | 23470.6 | 6.83 | 6.92 | 672.61 | 10.13 | 0.27 | 62.41 | 25.83 |
| 0.34 | 22557.5 | 6.46 | 6.36 | 627.80 | 11.51 | 0.12 | 32.57 | 35.05 |
| 0.89 | 20085.8 | 5.79 | 5.43 | 154.37 | 10.95 | 0.45 | 120.28 | 193.99 |
| 1.19 | 21400.6 | 5.93 | 5.58 | 375.04 | 8.87 | 0.25 | 139.55 | 66.77 |
| 0.01 | 19852.4 | 4.87 | 4.14 | 188.31 | 8.60 | 0.17 | 0 | 70.52 |
| 1.04 | 20683.1 | 6.56 | 0.00 | 292.65 | 13.93 | 2.88 | 100.13 | 77.2 |
| 0.01 | 20377.6 | 8.75 | 8.86 | 157.30 | 4.05 | 0.12 | 141.63 | 129.98 |
| 0.01 | 23779.6 | 9.13 | 9.16 | 163.75 | 2.13 | 0.2 | 56.52 | 52.56 |
| 0.25 | 25286.6 | 8.96 | 8.81 | 214.08 | 2.13 | 3.22 | 105.56 | 151.04 |
| 0.01 | 25437.7 | 8.71 | 8.07 | 83.87 | 1.74 | 0.16 | 70.73 | 53.93 |
| 0.01 | 22155.9 | 7.72 | 7.89 | 595.27 | 15.20 | 0.1 | 216.7 | 172.01 |
| 0.01 | 18918.6 | 7.24 | 7.05 | 564.08 | 35.01 | 0.54 | 141.41 | 144.68 |
| 0.01 | 23889.5 | 6.67 | 6.54 | 292.60 | 13.70 | 0.17 | 128.19 | 192.21 |
| 0.01 | 16728.5 | 7.25 | 8.61 | 331.95 | 11.95 | 0.22 | 146.87 | 51.97 |
| 0.01 | 26144.9 | 3.89 | 5.48 | 211.27 | 8.03 | 0.29 | 284.05 | 204.15 |
| 0.35 | 25853.1 | 4.43 | 6.03 | 155.64 | 11.84 | 0.41 | 413.44 | 255 |
| 0.01 | 26076.2 | 3.80 | 5.65 | 167.42 | 3.15 | 0.49 | 268.5 | 178.48 |
| 0.01 | 25932.0 | 4.00 | 6.27 | 214.07 | 6.20 | 0.15 | 215.71 | 181.5 |
| 0.61 | 23117.1 | 7.70 | 7.44 | 312.39 | 12.56 | 0.2 | 22.83 | 34.15 |
| 0.22 | 22008.2 | 8.14 | 7.94 | 261.47 | 12.23 | 0.04 | 14.84 | 71.08 |
| 0.01 | 23281.8 | 7.70 | 7.51 | 144.16 | 10.83 | 0.15 | 1.87 | 36.64 |
| 0.21 | 23827.7 | 8.56 | 8.60 | 347.21 | 11.20 | 0.23 | 37.69 | 81.78 |
| 0.19 | 13275.0 | 6.63 | 6.16 | 276.13 | 2.89 | 0.27 | 1.32 | 71.7 |
| 0.01 | 14267.1 | 5.67 | 5.64 | 201.88 | 4.06 | 0.22 | 5 | 72.2 |
| 0.01 | 13759.0 | 6.10 | 5.46 | 195.62 | 14.48 | 0.46 | 6.51 | 95.36 |
| 0.01 | 11922.4 | 5.89 | 7.26 | 173.52 | 5.95 | 0.33 | 9.46 | 109.03 |
| 0.31 | 23597.7 | 11.69 | 12.00 | 235.82 | 13.99 | 0.07 | 13.49 | 27.51 |
| 0.19 | 26103.7 | 12.34 | 12.61 | 134.30 | 10.50 | 0.08 | 20.62 | 69.97 |
| 0.65 | 23281.8 | 11.81 | 11.96 | 192.95 | 8.41 | 0.12 | 10.45 | 20.45 |
| 0.01 | 18602.8 | 12.23 | 12.54 | 131.20 | 12.63 | 0.18 | 35.16 | 19.93 |
| 0.28 | 16972.2 | 5.29 | 5.41 | 346.93 | 11.04 | 0.04 | 40.23 | 37.47 |
| 0.32 | 13978.7 | 5.33 | 5.83 | 411.55 | 8.52 | 0.37 | 106.25 | 143.69 |
| 0.01 | 19643.0 | 6.87 | 7.58 | 394.37 | 5.19 | 0.08 | 50.4 | 50.79 |
| 0.19 | 22358.4 | 5.64 | 6.19 | 496.21 | 5.71 | 0.1 | 53.85 | 31.58 |
| 0.01 | 32815.0 | 5.38 | 7.24 | 78.82 | 7.88 | 0.81 | 342.68 | 187.91 |
| 0.01 | 31562.0 | 5.66 | 6.80 | 84.60 | 5.61 | 0.22 | 464.91 | 203.49 |
| 0.01 | 31668.4 | 5.84 | 7.13 | 145.81 | 5.29 | 0.47 | 424.18 | 142.75 |
| 0.01 | 31517.3 | 5.54 | 7.72 | 135.27 | 7.56 | 0.48 | 457.78 | 199.5 |
| 0.01 | 17682.8 | 7.79 | 7.97 | 337.95 | 5.10 | 0.12 | 26.72 | 43.4 |
| 0.01 | 22598.7 | 11.00 | 8.73 | 442.81 | 19.39 | 0.19 | 41.54 | 88.32 |
| 0.01 | 29162.4 | 10.98 | 11.85 | 416.61 | 8.31 | 0.08 | 42.12 | 227.85 |
| 0.22 | 17555.8 | 5.05 | 6.72 | 549.47 | 8.17 | 0.06 | 18.67 | 84.22 |
| 0.22 | 19522.8 | 5.58 | 7.72 | 211.68 | 4.77 | 0.16 | 171.9 | 219.35 |
| 0.01 | 22406.5 | 14.43 | 15.61 | 312.20 | 17.97 | 0.21 | 36.4 | 153.07 |
| 0.01 | 28777.9 | 15.31 | 14.66 | 648.21 | 20.72 | 0.11 | 42.28 | 123.46 |
| 0.01 | 24383.8 | 8.71 | 9.72 | 161.75 | 7.51 | 0.12 | 9.31 | 37.18 |
| 0.01 | 25588.7 | 4.94 | 5.91 | 382.36 | 5.56 | 0.23 | 23.41 | 107.61 |
| 0.01 | 27655.3 | 19.66 | 20.94 | 430.28 | 24.59 | 0.23 | 36.29 | 19 |
| 8.96 | 33285.3 | 35.21 | 36.19 | 681.80 | 47.89 | 0.32 | 13.79 | 11.31 |
| 0.01 | 23175.4 | 11.24 | 11.77 | 245.77 | 4.88 | 0.09 | 17.54 | 29.76 |
| 58.68 | 25369.0 | 4.58 | 6.77 | 243.74 | 5.32 | 0.21 | 16.14 | 41.05 |
| 25.49 | 42626.2 | | | 715.32 | 161.89 | 6.14 | 951.94 | 189.61 |
| 26.21 | 43027.8 | | | 1168.94 | 182.08 | 2.14 | 540.35 | 76.54 |
| 26.90 | 43838.0 | | | 705.92 | 143.19 | 7.25 | 620.92 | 255.31 |
| 44.04 | 39495.4 | | | 577.25 | 107.21 | 23.02 | 512.21 | 102.22 |
| 40.13 | 40995.5 | | | 853.30 | 121.83 | 22.85 | 514.22 | 63.91 |
| 34.41 | 41325.1 | | | 373.23 | 87.69 | 22.36 | 517.81 | 88.06 |
| 0.33 | 62258.8 | | | 394.30 | 158.47 | 2.23 | 528.36 | 179.65 |
| 0.65 | 64974.3 | | | 577.49 | 178.66 | 2.53 | 949.62 | 229.55 |
| 0.15 | 41678.7 | | | 431.17 | 179.40 | 3.49 | 406.19 | 97.89 |
| 8.86 | 35732.9 | | | 276.49 | 12.99 | 18.5 | 1817.35 | 101.71 |
| 7.58 | 34737.4 | | | 131.15 | 99.61 | 15.29 | 1061.86 | 128.92 |
| 7.48 | 35338.1 | | | 190.92 | 107.58 | 62.22 | 850.98 | 167.84 |
| 0.09 | 42763.5 | | | 223.60 | 88.97 | 15.52 | 179.83 | 91.23 |
| 0.14 | 46210.1 | 7.00 | | 245.52 | 12.39 | 4.08 | 357.31 | 55.11 |

| TOCOPH*** | 66-3* | XAN/HVA | TRP/KYN | HX/XAN | TRY/4HPLA |
|---|---|---|---|---|---|
| 0.88 | 0 | 4.77 | 39.74 | 2.14 | 29.1818 |
| 0.35 | 1.08 | 4.84 | 61.48 | 0.50 | 23.4074 |
| 1.15 | 0 | 9.21 | 39.88 | 1.39 | 27.6334 |
| 1.7 | 0 | 5.18 | 64.62 | 1.13 | 28.5347 |
| 0.97 | 0 | 9.06 | 43.20 | 2.61 | 22.4507 |
| 5.18 | 0 | 3.17 | 29.01 | 0.53 | 16.7079 |
| 4.26 | 0.13 | 3.78 | 46.51 | 0.38 | 13.3094 |
| 1.22 | 0 | 8.59 | 27.56 | 1.75 | 20.9631 |
| 1.91 | 0 | 6.69 | 55.46 | 1.65 | 34.2647 |
| 4.67 | 0.03 | 11.80 | 29.78 | 0.25 | 21.2228 |
| 11.64 | 0 | | 20.47 | 2.12 | 46.0465 |
| 27.35 | 0.01 | | 19.02 | 1.75 | 35.2662 |
| 16.84 | 0.01 | | 19.17 | 1.57 | 40.6855 |
| 23.37 | 0 | | 19.30 | 1.28 | 49.094 |
| 69.65 | 1.25 | | 26.80 | 12.00 | 62.5527 |
| 57.69 | 1.39 | | 24.84 | 2.42 | 55.8338 |
| 0.07 | 0.02 | | 24.63 | 1.81 | 61.2709 |
| 51.55 | 1.33 | | 30.30 | 2.19 | 56.8523 |
| 71.34 | 0.01 | | 21.86 | 7.07 | 35.1551 |
| 34.14 | 0.01 | | 24.18 | 7.77 | 41.3027 |
| 86.56 | 0.01 | | 21.96 | 9.40 | 37.4434 |
| 58.61 | 0.01 | | 25.84 | 6.79 | 51.5344 |
| 75.67 | 0.01 | | 33.53 | 2.28 | 33.3012 |
| 92.95 | 0.02 | | 30.98 | 2.02 | 29.2721 |
| 80.73 | 0 | | 32.67 | 0.56 | 32.3085 |
| 48.24 | 0.02 | | 32.22 | 0.69 | 31.7918 |
| 71.16 | 0.01 | | 22.28 | 10.53 | 49.6184 |
| 101.86 | 0.01 | | 20.56 | 10.76 | 43.3515 |
| 63.08 | 0 | | 20.26 | 6.69 | 45.841 |
| 80.26 | 0 | | 20.71 | 6.52 | 41.2996 |
| 13.27 | 0 | | 35.11 | 7.57 | 57.0832 |
| 14.55 | 0 | | 31.18 | 4.15 | 37.6322 |
| 4.07 | 0 | | 30.96 | 3.30 | 44.6259 |
| 24.63 | 0 | | 32.40 | 7.34 | 49.3724 |
| 5.27 | 0.04 | | 43.18 | 5.01 | 45.7185 |
| 4.24 | 0.01 | | 41.27 | 2.41 | 39.9178 |
| 5.09 | 0.03 | | 40.09 | 2.80 | 44.653 |
| 5.72 | 0.04 | | 41.33 | 2.16 | 42.7986 |
| 22.01 | 0.01 | | 19.34 | 7.69 | 31.5351 |
| 52.61 | 0.01 | | 18.64 | 2.57 | 26.4739 |
| 14.39 | 0 | | 19.16 | 6.25 | 30.5533 |
| 25.39 | 0.01 | | 17.69 | 3.32 | 28.2274 |
| 57.99 | 0.02 | | 30.39 | 5.41 | 34.5109 |
| 163.72 | 0.01 | | 27.66 | 4.11 | 28.2506 |
| 59.22 | 0.02 | | 28.49 | 3.15 | 31.6218 |
| 41.1 | 0.02 | | 28.57 | 2.32 | 32.0854 |
| 47.11 | 0.03 | | 24.57 | 8.76 | 22.8239 |
| 89.48 | 0.03 | | 22.22 | 7.61 | 18.6024 |
| 53.89 | 0.03 | | 24.20 | 8.46 | 22.967 |
| 58.13 | 0.03 | | 23.97 | 7.90 | 22.2177 |
| 24.25 | 0.01 | | 24.78 | 4.62 | 41.1188 |
| 69.65 | 0.01 | | 20.83 | 0.58 | 30.9561 |
| 126.32 | 0.01 | | 15.29 | 3.45 | 22.1492 |
| 150.03 | 0.02 | | 29.11 | 0.80 | 36.7398 |
| 156.31 | 0.06 | | 22.26 | 4.61 | 30.8114 |
| 41.91 | 0.01 | | 19.95 | 2.17 | 25.1828 |
| 50.95 | 0.02 | | 17.56 | 0.15 | 31.8039 |
| 18.67 | 0.01 | | 14.52 | 1.40 | 22.6048 |
| 122.08 | 0 | | 19.61 | 3.06 | 45.7932 |
| 6.53 | 0 | | 9.86 | 1.88 | 21.7558 |
| 10.6 | 14.23 | | 5.80 | 0.09 | 9.44685 |
| 13.39 | 0 | | 15.43 | 7.00 | 23.0617 |
| 162.58 | 31.53 | | 20.07 | 5.17 | 46.8994 |
| 172.5 | 368.42 | | 13.07 | 3.54 | 40.9459 |
| 66.9 | 387.45 | | 13.55 | 3.16 | 43.8418 |
| 199.39 | 429.77 | | 13.58 | 2.93 | 43.5482 |
| 179.11 | 205.24 | | 21.69 | 4.12 | 42.259 |
| 205.04 | 326.07 | | 22.79 | 2.51 | 44.6527 |
| 158.97 | 347.47 | | 21.28 | 3.92 | 41.5429 |
| 192.5 | 0.04 | | 14.03 | 0.76 | 17.7592 |
| 84.68 | 0.08 | | 14.79 | 1.56 | 18.6863 |
| 90.75 | 0.07 | | 14.29 | 0.93 | 18.2126 |
| 269.07 | 75.8 | | 14.03 | 3.92 | 44.2056 |
| 160.89 | 95.87 | | 12.48 | 2.52 | 44.3876 |
| 134.85 | 76.93 | | 12.48 | 3.60 | 47.9083 |
| 50.96 | 0.31 | | 13.00 | 6.07 | 15.9826 |
| 63.79 | 0.31 | | 13.20 | 4.56 | 17.1648 |

| | | | | | | | | | |
|---|---|---|---|---|---|---|---|---|---|
| 0.11 | 47511.2 | | | 265.86 | 102.75 | 17.49 | 200.59 | 38.34 | 42.87 |
| 0.01 | 44002.7 | | | 472.13 | 164.85 | 376.81 | 429.78 | 125.66 | 68.88 |
| 0.17 | 44109.2 | | | 380.70 | 163.47 | 418.69 | 502.74 | 203.66 | 94.31 |
| 0.20 | 43233.8 | 5.29 | | 388.16 | 153.20 | 443.74 | 323.49 | 248.3 | 157.47 |
| 0.54 | 37772.1 | | | 334.54 | 100.72 | 2.72 | 554.64 | 159.45 | 121.28 |
| 0.46 | 36230.7 | | | 261.68 | 112.58 | 3.3 | 310.49 | 186.66 | 145.75 |
| 6.24 | 35187.1 | | | 461.67 | 157.47 | 4.32 | 756.44 | 176.33 | 132.58 |
| 0.52 | 42375.6 | | | 289.15 | 102.69 | 2.61 | 449.6 | 243.49 | 159.11 |
| 0.12 | 38702.4 | | | 243.07 | 105.61 | 1.14 | 1047.94 | 78.18 | 40.07 |
| 0.13 | 43405.4 | | | 253.99 | 90.00 | 3 | 849.83 | 230.24 | 180.26 |
| 106.03 | 52063.2 | | | 920.37 | 87.56 | 10.54 | 70.28 | 40.89 | 64.65 |
| 108.29 | 39516.0 | | | 593.18 | 112.46 | 11.48 | 127.81 | 35.33 | 63.4 |
| 101.16 | 53292.1 | | | 1109.63 | 95.54 | 9.75 | 115.93 | 22.47 | 69.17 |
| 0.01 | 37885.3 | | | 659.98 | 95.78 | 100 | 100 | 100 | 100 |
| 0.29 | 35001.7 | | | 300.33 | 70.90 | 101.88 | 47.6 | 137.9 | 147.73 |
| 0.31 | 35169.9 | | | 462.03 | 62.87 | 109.87 | 123.43 | 172.82 | 197.36 |
| 0.41 | 31870.9 | | | 578.20 | 81.57 | 3.44 | 212.96 | 213.24 | 175.16. |
| 0.13 | 34328.9 | | | 349.30 | 65.29 | 2.3 | 520.47 | 213.48 | 170.58 |
| 0.12 | 34933.1 | | | 371.33 | 68.78 | 4.53 | 249.83 | 277.27 | 195.73 |
| 13.94 | 48022.7 | | | 528.23 | 164.49 | 4.79 | 219.62 | 85.47 | 50.06 |
| 12.22 | 42145.6 | | | 501.42 | 134.14 | 5.46 | 206.17 | 68.35 | 38.32 |
| 13.78 | 47813.3 | | | 376.48 | 215.52 | 0.32 | 37.82 | 85.91 | 92.63 |
| 4.29 | 37298.3 | | | 497.32 | 70.90 | 129.09 | 11.73 | 17.28 | 12.84 |
| 4.18 | 34531.4 | | | 266.33 | 58.22 | 131.37 | 67.13 | 82.04 | 84.87 |
| 4.45 | 35478.9 | | | 387.53 | 58.72 | 290.96 | 51.92 | 55.99 | 64.96 |
| 1.01 | 40429.1 | | | 333.67 | 428.81 | 0.54 | 528.18 | 236.79 | 180.25 |
| 0.25 | 49183.0 | | | 1007.88 | 451.71 | 1.06 | 313.55 | 129.43 | 113.96 |
| 0.09 | 40322.7 | 32.53 | | 796.98 | 369.34 | 1.64 | 322.56 | 257.01 | 182.82 |
| 0.58 | 41719.9 | | | 570.99 | 93.77 | 40.06 | 115.03 | 88.95 | 99.28 |
| 0.44 | 41898.4 | | | 456.13 | 7.30 | 39.83 | 57.11 | 88.43 | 105.55 |
| 0.41 | 41129.4 | 11.02 | | 562.30 | 225.11 | 42.53 | 118.14 | 77.5 | 78.78 |
| 3.63 | 30281.5 | | | 256.19 | 90.98 | 16.69 | 372.85 | 268.04 | 89.49 |
| 4.66 | 30309.0 | | | 462.26 | 176.09 | 14.84 | 394.7 | 217.96 | 86.9 |
| 0.78 | 29313.4 | | | 313.16 | 136.12 | 12.55 | 690.97 | 271.56 | 116.63 |
| 0.06 | 33951.3 | | | 134.31 | 149.36 | 112.52 | 390.63 | 112.52 | 107.35 |
| 0.01 | 34033.6 | | | 103.38 | 117.43 | 115.11 | 335.3 | 271.7 | 119.4 |
| 0.17 | 33501.5 | | | 182.37 | 133.55 | 108.32 | 87.46 | 223.37 | 80.65 |
| 0.62 | 34455.9 | | | 809.58 | 2.15 | 1.38 | 205.5 | 160.51 | 85.33 |
| 0.01 | 28214.9 | | | 446.64 | 136.52 | 1.12 | 263.18 | 146.26 | 54.43 |
| 0.01 | 34198.4 | | | 737.24 | 109.83 | 0.53 | 233.21 | 210.69 | 111.04 |
| 0.62 | 30127.0 | 9.55 | | 183.74 | 130.37 | 51.33 | 80.22 | 181.77 | 29.3 |
| 1.14 | 31606.6 | | | 235.01 | 133.82 | 94.9 | 402.09 | 559.16 | 73.97 |
| 0.01 | 35719.2 | | | 85.96 | 162.55 | 334.73 | 232.36 | 80.74 | 69.26 |
| 0.18 | 34833.5 | | | 77.74 | 156.06 | 312.98 | 280.42 | 107.35 | 47.72 |
| 0.07 | 33824.2 | | | 164.28 | 180.32 | 318.97 | 129.9 | 125.15 | 53.98 |
| 5.65 | 28753.9 | | | 83.28 | 131.64 | 361.42 | 436.76 | 191.86 | 86.92 |
| 4.89 | 28595.9 | 8.22 | | 350.50 | 191.04 | 263.63 | 397.75 | 238.04 | 80.71 |
| 5.10 | 28877.4 | 9.03 | | 273.92 | 218.50 | 282.93 | 412.38 | 96.97 | 47.44 |
| 0.01 | 27665.6 | | | 474.77 | 132.73 | 115.06 | 251.98 | 314.37 | 75.7 |
| 0.01 | 27878.5 | | | 225.80 | | 118.21 | 208.83 | 265.12 | 78.32 |
| 0.45 | 26670.1 | | | 142.04 | 115.45 | 593.76 | 146.97 | 102.66 | 64.52 |
| 0.01 | 39361.5 | 4.58 | 5.46 | 624.78 | 220.6 | 2.47 | 31.83 | 60.01 | 41.86 |
| 0.01 | 38695.5 | 4.54 | 3.25 | 358.94 | 189.25 | 0.75 | 125.04 | 79.62 | 134.59 |
| 0.01 | 39165.8 | 4.72 | 5.18 | 443.55 | 115.32 | 0.67 | 65 | 22.07 | 56.03 |
| 1.04 | 33312.7 | 6.55 | 6.96 | 92.84 | 72.71 | 0.68 | 294.02 | 70.36 | 143.73 |
| 0.01 | 35990.4 | 5.49 | 7.33 | 222.27 | 35.56 | 48.18 | 19.25 | 50.99 | 33.25 |
| 0.01 | 36289.0 | 4.93 | 6.19 | 266.87 | 46.42 | 41.26 | 14.38 | 52.64 | 48.83 |
| 0.01 | 35705.5 | 4.54 | 6.55 | 239.96 | 67.31 | 54.31 | 21.65 | 39.23 | 36.93 |
| 0.01 | 32650.2 | 2.84 | 8.38 | 247.77 | 60.42 | 1.75 | 70.6 | 27.9 | 135.36 |
| 0.01 | 26354.3 | 2.57 | 4.79 | 343.64 | 64.71 | 1.59 | 11.56 | 35.9 | 51.15 |
| 0.01 | 30700.3 | 3.01 | 5.9 | 292.89 | 39.27 | 0.86 | 66.67 | 65.03 | 101.79 |
| 0.17 | 3597.41 | | | 241.73 | 93.29 | 0.57 | 10.34 | 0 | 1.76 |
| 0.01 | 3453.33 | | | 320.45 | 140.29 | 2.39 | | 0 | 0.21 |
| 3.08 | 4479.32 | | | 298.96 | 150.09 | 3.9 | | 0.1 | 0 |
| 0.41 | 5384.11 | | | 409.67 | 238.98 | 4.04 | 1.82 | 0 | 0.2 |
| 0.01 | 6623.51 | | | 198.73 | 115.97 | 3.53 | 6.41 | 0 | 0.74 |
| 0.11 | 1931.93 | | | 119.54 | 80.47 | 0.75 | 1.5 | 0 | 0.19 |
| 0.55 | 4752.98 | | | 361.55 | 112.76 | 1.42 | 14.49 | 1.81 | 0.03 |
| 1.45 | 5677.47 | | | 291.75 | 107.52 | 1.09 | 57.82 | 4.28 | 1.78 |
| 0.05 | 6310.14 | | | 440.03 | 90.82 | 1.08 | 12.98 | 1.09 | 1.21 |
| 0.01 | 6717.97 | | | 253.35 | 99.99 | 0.3 | 29.95 | 6.92 | 1.79 |
| 0.05 | 5670.72 | | | 617.07 | 130.09 | 1.63 | 125.08 | 27.05 | 2.1 |
| 1.28 | 7853.13 | | | 195.51 | 53.97 | 0.44 | 7.41 | 1.5 | 0.54 |
| 0.01 | 3948.84 | | | 298.39 | 92.65 | 0.72 | 11.52 | 1.61 | 1.3 |
| 0.05 | 3647.74 | | | 447.17 | 21.11 | 0.46 | 21.26 | 4.64 | 1.96 |
| 0.01 | 5691.31 | | | 357.9 | 161.6 | 0.41 | 14.49 | 3.91 | 1.07 |
| 0.07 | 5166 | | | 439.4 | 138.75 | 0.29 | 6.66 | 3.81 | 1.04 |
| 0.29 | 4783.28 | 0.00 | 0.00 | 316.42 | 74.80 | 0.96 | 26.07 | 11.73 | 1.47 |

| | | | | |
|---|---|---|---|---|
| 0.33 | | 14.08 | 5.11 | 16.4322 |
| 0.16 | | 14.78 | 2.68 | 24.4696 |
| 0.16 | | 14.91 | 2.40 | 23.8869 |
| 0.16 | | 14.68 | 2.19 | 23.3094 |
| 3.19 | | 13.85 | 3.83 | 38.2388 |
| 3 | | 14.40 | 5.24 | 40.5567 |
| 2.15 | | 13.77 | 4.75 | 43.1521 |
| 0.27 | | 23.95 | 4.57 | 19.0257 |
| 0.19 | | 22.83 | 3.55 | 15.9819 |
| 0.27 | | 23.36 | 4.33 | 17.8377 |
| 192.21 | | 10.58 | 0.83 | 24.7597 |
| 163.56 | | 10.56 | 0.50 | 25.8061 |
| 161.42 | | 10.84 | 2.43 | 26.748 |
| 0.14 | | 27.90 | 4.22 | 39.5098 |
| 0.15 | | 26.91 | 2.57 | 40.7588 |
| 0.15 | | 28.42 | 2.14 | 36.5895 |
| 0.14 | | 16.71 | 2.19 | 18.7663 |
| 0.14 | | 16.32 | 1.36 | 23.3312 |
| 0.14 | | 16.48 | 1.36 | 23.2888 |
| 108.9 | | 10.74 | 4.38 | 31.2038 |
| 100 | | 11.22 | 3.98 | 29.8557 |
| 95.64 | | 11.19 | 2.40 | 25.0533 |
| 14.64 | | 24.75 | 4.53 | 33.6665 |
| 13.98 | | 23.24 | 3.12 | 25.2294 |
| 62.83 | | 26.25 | 4.68 | 40.2681 |
| 0.14 | | 9.86 | 0.46 | 7.4968 |
| 0.14 | | 10.02 | 1.54 | 8.80645 |
| 0.14 | | 10.40 | 0.80 | 7.89757 |
| 0.35 | | 16.24 | 3.72 | 28.4863 |
| 0.26 | | 16.77 | 3.05 | 27.9863 |
| 0.4 | | 15.23 | 3.84 | 27.7011 |
| 16.97 | | 18.04 | 3.28 | 39.5244 |
| 16.44 | | 19.25 | 5.95 | 44.4469 |
| 14.49 | | 17.77 | 5.88 | 38.5408 |
| 0.08 | | 16.10 | 2.64 | 32.584 |
| 0.07 | | 14.60 | 3.47 | 39.192 |
| 0.07 | | 13.96 | 4.87 | 34.8286 |
| 0.08 | | 25.22 | 4.30 | 28.1323 |
| 0.06 | | 24.11 | 3.02 | 27.438 |
| 0.08 | | 25.09 | 4.02 | 36.453 |
| 0.08 | | 17.44 | 8.04 | 36.6539 |
| 0.17 | | 18.04 | 8.36 | 41.9666 |
| 0.08 | | 14.72 | 2.57 | 37.1724 |
| 0.01 | | 15.34 | 3.57 | 39.24 |
| 0.07 | | 14.26 | 5.14 | 36.3628 |
| 0.41 | | 12.35 | 2.69 | 30.3285 |
| 0.28 | | 13.12 | 6.18 | 40.2597 |
| 0.32 | | 12.49 | 6.30 | 31.9487 |
| 0.01 | | 18.62 | 6.15 | 43.0068 |
| 0.08 | | 20.93 | 9.08 | 46.0207 |
| 0.07 | | 21.10 | 5.64 | 45.7603 |
| 0.02 | | | | |
| 0 | | | | |
| 0 | | | | |
| 0.04 | | | | |
| 0.03 | | | | |
| 0.02 | | | | |
| 0.01 | | | | |
| 0.05 | | | | |
| 0.04 | | | | |
| 0.01 | | | | |
| 0.01 | 9.53 | 43.75 | 1.61 | 16.9441 |
| 0 | 6.35 | 22.70 | 2.16 | 38.409 |
| 0.3 | 6.72 | 38.07 | 2.36 | 19.9897 |
| 0 | 13.94 | 47.01 | 1.79 | 27.3269 |
| 0 | 12.32 | 49.24 | 1.70 | 36.7868 |
| 0 | 7.07 | 46.79 | 2.52 | 31.22 |
| 132.19 | 8.62 | 37.80 | 1.54 | 23.5226 |
| 253.36 | 9.03 | 57.09 | 1.33 | 39.5784 |
| 0.32 | 12.07 | 57.82 | 1.78 | 23.2443 |
| 0.02 | 9.68 | 32.60 | 0.98 | 20.2941 |
| 2.33 | 12.59 | 50.69 | 0.69 | 17.5387 |
| 118.58 | 19.02 | 25.39 | 0.99 | 45.4888 |
| 0.05 | 10.06 | 68.99 | 1.75 | 39.0901 |
| 0.02 | 12.90 | 62.65 | 1.62 | 25.5882 |
| 0.01 | 11.29 | 45.04 | 1.86 | 16.3389 |
| 0.32 | 10.89 | 37.69 | 1.15 | 26.7326 |
| 19.57 | 9.20 | 43.87 | 1.47 | 26.38 |

| | | | | | | | | |
|---|---|---|---|---|---|---|---|---|
| 0.68221 | 1667.4123 | 0 | 0 | 102.8429787 | 63.45958021 | 1.19022 | 41.5086 | 34.73090794 |
| 26 | 26 | 10 | 10 | 26 | 26 | 26 | 24 | 26 |
| 0 | 2099.3 | | | 335.96 | 122.78 | 0 | 0 | 0 |
| 20 | 100000 | | | 200 | 1000 | | | |
| 20.41 | 9324.3 | | | 202.94 | 1063.92 | | | |
| 1.44 | 22751.17 | 8.20 | 8.55 | 307.60 | 10.51 | 0.42 | 100.64 | 98.37 |
| 8.10925 | 4967.8259 | 4.9171 | 5.0432 | 168.22 | 8.0200 | 0.8286 | 125.1962 | 67.8534 |
| 53 | 53 | 53 | 53 | 53 | 52 | 53 | 53 | 53 |
| 9.71 | 38528.88 | 11.81 | | 424.42 | 133.10 | 88.52 | 381.62 | 156.16 |
| 23.3205 | 7703.0647 | 9.3212 | | 248.72 | 80.47 | 136.34 | 315.79 | 91.75 |
| 65 | 65 | 7 | | 65 | 64 | 65 | 65 | 65 |

| | | | | | | |
|---|---|---|---|---|---|---|
| 1.354758962 | 58.57601752 | 3.597252662 | 12.96442212 | 0.651942931 | 8.39928 | |
| 26 | 26 | 26 | 26 | 26 | 26 | 0 |
| 14.2 | 0 | 6.42 | 42.36 | 1.67 | | |
| | | | 10.00 | 0.50 | | |
| | | | 9.36 | 0.73 | | |
| 53.55 | 0.95 | | 24.66 | 4.41 | 36.98 | |
| 43.5550 | 4.7110 | | 7.691401564 | 3.066954008 | 11.6657 | |
| 53 | 53 | | 53 | 53 | 53 | |
| 107.99 | 50.60 | | 16.74 | 3.66 | 31.49 | |
| 54.34 | 106.07 | | 5.006705677 | 1.847796061 | 10.6805 | |
| 65 | 65 | | 65 | 65 | 65 | |

TABLE BC-BY

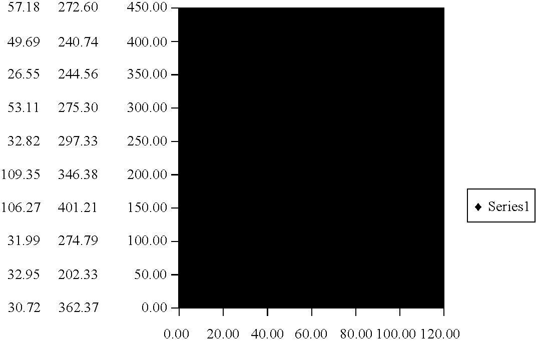

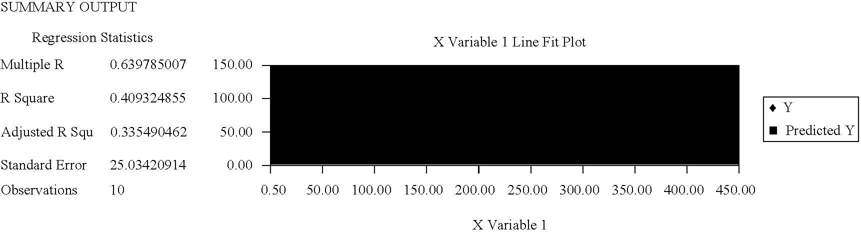

ANOVA

| | df | SS | MS | F | Significance F | | | |
|---|---|---|---|---|---|---|---|---|
| Regression | 1 | 3474.378738 | 3474.378738 | 5.543823646 | 0.046352134 | | | |
| Residual | 8 | 5013.693017 | 626.7116271 | | | | | |
| Total | 9 | 8488.071755 | | | | | | |
| | Coefficients | Standard Error | tStat | P-value | Lower 95% | Upper 95% | Lower 95.0% | Upper 95.0% |
| Intercept | 45.52367885 | 42.61283134 | 1.068309179 | 0.316554047 | 143.7891077 | 52.74174999 | 143.7891077 | 52.74174999 |
| X Variable 1 | 0.3358303 | 0.14263141 | 2.354532575 | 0.046352134 | 0.006921467 | 0664739133 | 0.006921467 | 0.664739133 |

RESIDUAL OUTPUT

| Observation | Predicted Y | Residuals |
|---|---|---|
| 1 | 46.02366297 | 11.15633734 |
| 2 | 35.32410941 | 14.36588922 |
| 3 | 36.60697849 | 10.05697925 |
| 4 | 46.93039863 | 6.179601982 |
| 5 | 70.80122209 | 38.54877639 |
| 7 | 89.21479293 | 17.05520372 |
| 8 | 46.75913214 | 14.76913237 |
| 9 | 28.46981175 | 4.480189008 |
| 10 | 76.17114531 | 45.45114599 |

TABLE BZ-DD

| | 2HPAC | 3OHAN | 3OHKY | 3OMD | 4HBAC | 4HPAC | 4HPLA | 5HIAA | 5HT |
|---|---|---|---|---|---|---|---|---|---|
| CSF1_I | 0.23 | 0.00 | 0.51 | 4.39 | 1.72 | 14.41 | 68.85 | 34.19 | 0.00 |
| PLASMA_1_I_AD_ | 0.27 | 0.46 | 3.33 | 17.73 | 2.73 | 84.86 | 127.51 | 4.68 | 40.5 |
| PLASMA_1_I_CVD_ | 0.00 | 0.00 | 2.80 | 16.44 | 3.15 | 92.44 | 166.47 | 4.32 | 20.84 |
| PLASMA_1_I_VD_ | 0.00 | 0.00 | 4.00 | 15.22 | 2.58 | 98.37 | 157.38 | 5.46 | 41.22 |
| PLASMA_1_I_VS | 0.00 | 0.00 | 3.85 | 17.77 | 3.95 | 95.80 | 139.45 | 5.55 | 39.15 |
| CSF1_II | 0.14 | 0.00 | 0.47 | 1.41 | 0.98 | 6.30 | 56.01 | 33.98 | 0.00 |
| PLASMA_1_II_AD | 1.45 | 1.16 | 6.74 | 12.93 | 2.86 | 47.83 | 111.64 | 7.41 | 0.67 |
| PLASAM_1_II_CVD | 1.84 | 1.06 | 7.13 | 16.14 | 5.06 | 47.87 | 131.39 | 7.60 | 4.07 |
| PLASMA_1_II_VD | 0.99 | 0.00 | 0.00 | 13.32 | 3.09 | 43.07 | 115.00 | 1.57 | 1.39 |
| PLASMA_1_II_VS | 1.46 | 2.43 | 7.94 | 14.69 | 7.22 | 47.23 | 125.28 | 8.33 | 1.79 |
| CSF1_III | 0.00 | 0.00 | 0.53 | 2.26 | 0.51 | 6.87 | 54.66 | 16.92 | 0.00 |
| PLASMA_1_III_AD | 0.00 | 0.62 | 4.29 | 13.60 | 1.93 | 62.37 | 179.81 | 9.62 | 17.57 |
| PLASMA_1_III_AS | 0.00 | 0.84 | 4.18 | 14.53 | 1.64 | 61.61 | 157.71 | 9.70 | 21.80 |
| PLASMA_1_III_CVD | 0.00 | 1.38 | 4.05 | 13.42 | 2.13 | 63.04 | 187.59 | 9.95 | 31.48 |
| PLASMA_1_III_VD | 3.12 | 0.36 | 3.01 | 12.51 | 2.61 | 64.39 | 143.45 | 8.58 | 1.59 |
| CSF1_IV | 0.49 | 0.00 | 0.46 | 4.24 | 0.49 | 34.00 | 113.97 | 36.70 | 0.00 |
| PLASMA_1_IV_AD | 5.26 | 2.06 | 2.67 | 14.38 | 2.47 | 249.98 | 301.38 | 7.08 | 33.36 |
| PLASMA_1_IV_CVD | 0.00 | 1.33 | 2.35 | 13.03 | 2.25 | 244.75 | 355.96 | 6.81 | 39.88 |
| PLASMA_1_IV_VD | 3.50 | 0.35 | 1.69 | 11.58 | 1.73 | 205.85 | 297.25 | 5.99 | 8.60 |
| PLASMA_1_IV_VS | 3.69 | 1.72 | 2.57 | 14.01 | 2.10 | 225.32 | 306.01 | 6.73 | 18.55 |
| CSF1_V | 1.00 | 0.00 | 0.75 | 3.30 | 0.34 | 6.71 | 50.03 | 21.40 | 0.00 |
| PLASMA_1_V_ARTERIA | 0.00 | 1.24 | 3.82 | 13.79 | 0.77 | 23.43 | 86.68 | 11.87 | 187.39 |
| PLASMA_1_V_CVD | 0.00 | 1.40 | 3.82 | 13.82 | 0.53 | 21.11 | 97.72 | 10.79 | 101.89 |
| PLASMA_1_V_V.SINIST | 0.00 | 1.30 | 4.15 | 14.22 | 0.82 | 21.23 | 91.97 | 11.29 | 171.60 |
| PLASMA_1_V_V.DEXTR | 0.00 | 1.45 | 4.08 | 14.33 | 1.03 | 23.06 | 101.07 | 11.77 | 279.06 |

| | 5HTOL | 5HTP | AM | ASC | CYS | DA** | DOPAC | EPI | G | GR |
|---|---|---|---|---|---|---|---|---|---|---|
| CSF1_I | | 1.17 | | 0.00 | | 17.87 | | 0.02 | 0.00 | 13.80 |
| PLASMA_1_I_AD_ | | 0.22 | 6.98 | 1.24 | | 0 | 0.15 | 9.77 | 0.77 | 4.43 |
| PLASMA_1_I_CVD_ | | 0.08 | | 0.00 | | 1.72 | | 0.04 | 0.00 | 18.30 |
| PLASMA_1_I_VD_ | | 0.21 | | 0.00 | | 0.11 | | 0.04 | 0.00 | 4.74 |
| PLASMA_1_I_VS | | 0.25 | | 0.00 | | 1.64 | | 0.04 | 0.00 | 1.70 |
| CSF1_II | | 1.29 | | 0.00 | | 17.63 | | 0.02 | 0.00 | 11.29 |
| PLASMA_1_II_AD | | 0.25 | | 0.00 | | 2.03 | | 0.04 | 0.45 | 12.80 |
| PLASAM_1_II_CVD | | 0.42 | | 0.00 | | 5.47 | | 0.04 | 1.40 | 43.92 |
| PLASMA_1_II_VD | | 0.91 | | 0.00 | | 5.17 | | 0.04 | 0.84 | 33.07 |
| PLASMA_1_II_VS | | 0.53 | | 0.00 | | 5.43 | | 0.04 | 0.41 | 10.25 |
| CSF1_III | | 0.78 | | 0.00 | | 20.04 | | 0.02 | 0.00 | 9.14 |
| PLASMA_1_III_AD | | 0.25 | | 0.00 | | 7.46 | | 0.04 | 0.90 | 28.34 |
| PLASMA_1_III_AS | | 0.17 | | 0.00 | | 6.22 | | 0.04 | 0.00 | 4.82 |
| PLASMA_1_III_CVD | | 0.18 | | 0.00 | | 6.19 | | 0.04 | 0.64 | 65.95 |
| PLASMA_1_III_VD | | 0.12 | | 0.00 | | 6.39 | | 0.04 | 0.31 | 34.61 |
| CSF1_IV | | 1.29 | | 0.00 | | 13.59 | | 0.02 | 0.00 | 9.79 |
| PLASMA_1_IV_AD | | 0.15 | | 0.00 | | 5.80 | | 0.04 | 0.70 | 56.28 |
| PLASMA_1_IV_CVD | | 0.14 | | 0.00 | | 5.10 | | 0.04 | 0.00 | 16.47 |
| PLASMA_1_IV_VD | | 0.22 | | 0.00 | | 4.73 | | 0.04 | 0.00 | 6.30 |
| PLASMA_1_IV_VS | | 0.31 | | 0.00 | | 5.86 | | 0.04 | 0.00 | 2.86 |
| CSF1_V | | 0.83 | | 5798.78 | | 19.92 | | 0.02 | 0.00 | 11.02 |
| PLASMA_1_V_ARTERIA | | 0.36 | | 0.00 | | 4.30 | | | 4.73 | 72.26 |
| PLASMA_1_V_CVD | | 0.23 | | 0.00 | | 1.51 | | | 2.53 | 31.49 |
| PLASMA_1_V_V.SINIST | | 0.13 | | 0.00 | | 3.95 | | | 1.55 | 33.49 |
| PLASMA_1_V_V.DEXTR | | 0.23 | | 0.00 | | 1.30 | | 0.04 | 1.76 | 32.69 |

| | GSH | GSSG | HGA | HVA | HX | KYN | LD | MEL | MET | MHPG |
|---|---|---|---|---|---|---|---|---|---|---|
| CSF1_I | | 0.00 | 0.00 | 57.18 | 583.50 | 11.15 | | 0.33 | 392.86 | 19.16 |
| PLASMA_1_I_AD_ | 0.32 | 19.2 | 0.01 | 11.09 | 1239.33 | 290.5 | 1.51 | 4.28 | 2275.28 | 10.75 |
| PLASMA_1_I_CVD_ | | 11.90 | 0.00 | 11.73 | 859.81 | 320.72 | | 9.18 | 2432.20 | 11.68 |
| PLASMA_1_I_VD_ | | 19.16 | 0.00 | 12.69 | 1059.18 | 375.53 | | 0.00 | 2670.00 | 12.51 |
| PLASMA_1_I_VS | | 22.66 | 0.00 | 14.33 | 804.67 | 355.00 | | 6.24 | 2460.51 | 12.48 |
| CSF1_II | | 0.00 | 0.00 | 49.69 | 119.29 | 14.70 | | 0.17 | 421.20 | 11.68 |
| PLASMA_1_II_AD | | 28.13 | 0.00 | 23.22 | 1852.76 | 421.67 | | 0.00 | 2875.50 | 7.79 |
| PLASAM_1_II_CVD | | 28.72 | 0.00 | 24.36 | 907.21 | 443.77 | | 0.69 | 3191.15 | 8.78 |
| PLASMA_1_II_VD | | 19.09 | 0.00 | 22.20 | 340.90 | 409.13 | | 0.98 | 2964.12 | 8.44 |
| PLASMA_1_II_VS | | 12.91 | 0.00 | 24.41 | 641.44 | 398.07 | | 3.70 | 2778.73 | 8.68 |
| CSF1_III | | 0.00 | 0.00 | 26.55 | 339.86 | 9.33 | | 0.25 | 575.60 | 15.10 |
| PLASMA_1_III_AD | | 6.09 | 0.00 | 24.85 | 1112.36 | 290.60 | | 8.58 | 2631.66 | 7.98 |
| PLASMA_1_III_AS | | 41.51 | 0.00 | 21.45 | 1271.94 | 255.09 | | 9.62 | 2623.39 | 7.65 |
| PLASMA_1_III_CVD | | 46.34 | 0.00 | 21.73 | 2012.91 | 342.13 | | 9.99 | 3041.91 | 8.56 |
| PLASMA_1_III_VD | | 31.60 | 0.00 | 20.37 | 569.13 | 245.44 | | 7.41 | 3733.12 | 6.65 |
| CSF1_IV | | 0.00 | 0.00 | 53.11 | 310.74 | 9.78 | | 0.50 | 824.12 | 18.80 |
| PLASMA_1_IV_AD | | 31.88 | 0.00 | 15.21 | 1359.38 | 263.42 | | 2.13 | 3267.55 | 7.39 |
| PLASMA_1_IV_CVD | | 29.36 | 0.00 | 15.56 | 1138.60 | 314.83 | | 0.54 | 3637.21 | 8.76 |
| PLASMA_1_IV_VD | | 40.10 | 0.00 | 13.66 | 165.14 | 245.49 | | 0.54 | 3313.26 | 7.95 |
| PLASMA_1_IV_VS | | 49.90 | 0.00 | 15.32 | 229.69 | 255.63 | | 0.75 | 3376.09 | 8.02 |
| CSF1_V | | 0.00 | 0.00 | 32.82 | 776.66 | 8.33 | | 0.42 | 549.45 | 12.94 |
| PLASMA_1_V_ARTERIA | | 17.14 | 0.65 | 7.84 | 2224.89 | 228.98 | | 0.28 | 2826.51 | 4.79 |
| PLASMA_1_V_CVD | | 43.96 | 0.64 | 7.85 | 1674.72 | 253.12 | | 1.01 | 2828.99 | 5.67 |
| PLASMA_1_V_V.SINIST | | 39.36 | 0.44 | 8.94 | 1119.25 | 274.31 | | 0.95 | 2795.28 | 5.57 |
| PLASMA_1_V_V.DEXTR | | 23.54 | 0.47 | 8.65 | 1396.36 | 298.79 | | 0.72 | 2773.82 | 6.02 |

TABLE DE-EL

| TPOL | TRP | TRYPT | TYR | TYRA | NA5HT | NE | NM5HT | URIC |
|---|---|---|---|---|---|---|---|---|
| | 443.10 | | 2009.17 | | | | 0 | 3166.05 |
| | 5948.0 | 43.29 | 5871.4 | 0.97 | 1.15 | | 0.48 | 21905.3 |
| | 6098.8 | | 5870.8 | | | | 0 | 20552.7 |
| | 7200.0 | | 6403.1 | | | | 0 | 23470.6 |
| | 6851.4 | | 6846.2 | | | | 0.34 | 22557.5 |
| | 903.77 | | 1311.05 | | | | 0 | 2550.12 |
| | 11301.2 | | 6983.4 | | | | 0.89 | 20085.8 |
| | 11022.5 | | 7336.0 | | | | 1.19 | 21400.6 |
| | 10076.9 | | 7046.2 | | | | 0.01 | 19852.4 |
| | 12060.7 | | 7122.5 | | | | 1.04 | 20683.1 |
| | 372.10 | | 1510.44 | | | | 0 | 2752.43 |
| | 6353.8 | | 6321.2 | | | | 0.01 | 20377.6 |
| | 6167.6 | | 6513.8 | | | | 0.01 | 23779.6 |
| | 7513.91 | | 7024.0 | | | | 0.25 | 25286.6 |
| | 6341.0 | | 7392.6 | | | | 0.01 | 25437.7 |
| | 631.97 | | 3252.10 | | | | 0 | 5936.43 |
| | 8831.2 | | 10036.3 | | | | 0.01 | 22155.9 |
| | 9753.8 | | 10419.7 | | | | 0.01 | 18918.6 |
| | 8019.1 | | 9603.7 | | | | 0.01 | 23889.5 |
| | 8235.8 | | 9728.6 | | | | 0.01 | 16728.5 |
| | 359.85 | | 1123.21 | | | | 0 | 2823.94 |
| | 5101.71 | | 4300.9 | | | | 0.01 | 26144.9 |
| | 5205.2 | | 4236.3 | | | | 0.35 | 25853.1 |
| | 5558.4 | | 4216.0 | | | | 0.01 | 26076.2 |
| | 6189.0 | | 4174.2 | | | | 0.01 | 25932.0 |

| VMA1 | VMA2 | XAN | ANTHOSIN | 84* | 86* | 96* | TOCOPH** | 66-3* |
|---|---|---|---|---|---|---|---|---|
| 0.00 | 0.00 | 272.60 | 12.56 | 0.14 | 7.69 | 0.95 | 0.88 | 0 |
| 7.02 | 6.9 | 585.16 | | 0.36 | 37.5 | 23.26 | 11.64 | 0 |
| 6.17 | 5.85 | 490.53 | 10.75 | 4.76 | 36.74 | 61.15 | 27.35 | 0.01 |
| 6.83 | 6.92 | 672.61 | 10.13 | 0.27 | 62.41 | 25.83 | 16.84 | 0.01 |
| 6.46 | 6.36 | 627.80 | 11.51 | 0.12 | 32.57 | 35.05 | 23.37 | 0 |
| 0.00 | 0.00 | 240.74 | 8.10 | 0.06 | 3.3 | 0.25 | 0.35 | 1.08 |
| 5.79 | 5.43 | 154.37 | 10.95 | 0.45 | 120.28 | 193.99 | 69.65 | 1.25 |
| 5.93 | 5.58 | 375.04 | 8.87 | 0.25 | 139.55 | 66.77 | 57.69 | 1.39 |
| 4.87 | 4.14 | 188.31 | 8.60 | 0.17 | 0 | 70.5 | 0.07 | 0.02 |
| 6.56 | 0.00 | 292.65 | 13.93 | 2.88 | 100.13 | 77.2 | 51.55 | 1.33 |
| 0.00 | 0.00 | 244.56 | 11.93 | 0.16 | 5.36 | 1.16 | 1.15 | 0 |
| 8.75 | 8.86 | 157.30 | 4.05 | 0.12 | 141.63 | 129.98 | 71.34 | 0.01 |
| 9.13 | 9.16 | 163.75 | 2.13 | 0.2 | 56.52 | 52.56 | 34.14 | 0.01 |
| 8.96 | 8.81 | 214.08 | 2.13 | 3.22 | 105.56 | 151.04 | 86.56 | 0.01 |
| 8.71 | 8.07 | 83.87 | 1.74 | 0.16 | 70.73 | 53.93 | 58.61 | 0.01 |
| 0.00 | 0.00 | 275.30 | 11.36 | 0.3 | 7.43 | 3.33 | 1.7 | 0 |
| 7.72 | 7.89 | 595.27 | 15.20 | 0.1 | 216.7 | 172.01 | 75.67 | 0.01 |
| 7.24 | 7.05 | 564.08 | 35.01 | 0.54 | 141.41 | 144.68 | 92.95 | 0.02 |
| 6.67 | 6.54 | 292.60 | 13.70 | 0.17 | 128.19 | 192.21 | 80.73 | 0 |
| 7.25 | 8.61 | 331.95 | 11.95 | 0.22 | 146.87 | 51.97 | 48.24 | 0.02 |
| 0.00 | 0.00 | 297.33 | 12.12 | 0.25 | 4.86 | 3.68 | 0.97 | 0 |
| 3.89 | 5.48 | 211.27 | 8.03 | 0.29 | 284.05 | 204.15 | 71.16 | 0.01 |
| 4.43 | 6.03 | 155.64 | 11.84 | 0.41 | 413.44 | 255 | 101.86 | 0.01 |
| 3.80 | 5.65 | 167.42 | 3.15 | 0.49 | 268.5 | 178.48 | 63.08 | 0 |
| 4.00 | 6.27 | 214.07 | 6.20 | 0.15 | 215.71 | 181.5 | 80.26 | 0 |

1. A method and procedure for combining assays of overall biochemical profiles of EC arrays with assays of carbon column switching of specific markers of genomic and proteomic interactions with those profiles to determine metabolic differences in stroke.

2. A method as in claim 1, wherein a peak suppressing gradient mixer is used to allow definition analytes in areas of the profile that would otherwise be obscured but contaminants.

3. A method for comparing the data obtained by protocols of claim 1 for presence or absence of both known and unknown compounds in a cohort of stroke subjects or controls.

4. A method as in claim 1, wherein a portion of the effluent of the columns from the EC arrays is diverted on line for structural identification of structurally unknown compounds.

5. A method according to claim 4, wherein the structural identification technique is mass spectroscopy

6. A method for separating DNA from biological samples for assay of patterns of ligands and associated compounds comprising: Disruption of the cellular material: crude separation of the DNA by molecular weight filters; preparation of aliquots of the crude extract by protocols of extraction, nuclease and/or or phosphatase digention, ligand competition or chemical digestion; and assay of the preparations by electrochemical arrays or carbon column switching.

7. A method of analyzing the data obtained according to claim 6 of comparison of the different preparation protocols for weak and strong ligands to DNA and associated proteins.

8. Methylation as an abnormal process in stroke.

9. Methylated and N methylated compounds as markers of stroke.

10. Methylated and N methylated compounds as predictors of outcome of stroke.

11. Methylated and N methylated compounds as monitors of therapy in stroke.

12. N methyl and NN dimethyl serotonins as predictors of outcome of stroke.

13. N methyl and NN dimethyl serotonins as therapy monitoring markers in stroke.

14. Methylation processes as target systems for therapeutic agents in stroke.

15. Compounds that suppress N methylation as therapeutic agents in stroke.

16. N methyl accepting compounds as therapeutic agents in stroke.

17. Compounds that suppress the formation of N methyl serotonins as therapeutic agents in stroke.

18. Shifts in the ratios of oxidative DNA damage markers 8OH2′dG and 8OHG and methylation or methylation protection markers 7MG of DNA as predictive and therapy monitoring markers in stroke.

19. A compound with coordinates in an electrochemical array with coordinates of 66 min dominant on ch3 as a predictive marker in stroke.

20. A group of compounds with coordinates in an electrochemical array of 84/4, 86/5 and 96/3 as therapeutic agents in stroke.

21. Combinatorial modifications of the group of compounds of claim 15 as therapeutic agents in stroke.

22. Combinatorial modifications of the group of compounds of claim 16 as therapeutic agents in stroke.

23. Combinatorial modifications of the group of compounds of claim 20 as therapeutic agents in stroke.

24. A group of compounds associated with DNA isolated from stroke subject samples with coordinates in an electrochemical array of 68-76 min ch9 and 10 as therapeutic targets in stroke.

25. Compounds that replace DNA ligands of the compound group of claim 24 as therapeutic agents in stroke.

26. Abnormalities in the Kynurinine pathway in stroke.

27. Long term abnormalities in the kynurinine pathway in stroke.

28. The kynurinine pathway as a therapeutic target for stroke.

29. Abnormalities in the Purine pathway in stroke.

30. The purine pathway as a therapeutic target in stroke.

31. A method of developing and selecting therapeutic approaches for the use of existing accepted drugs for short term therapy and prevention of long term sequellae of stroke comprising; analysis of samples for subjects post recovery from a stroke and creation of a database according to claim 1; analyzing the database for pathways contributing to the biochemical separation of the group; defining the biochemistry of model systems analyzed according to claim 1; testing of compounds that modulate these pathways in these defined cell, invertebrate and vertebrate models; comparing the pathway effects across the model and human data bases.

32. A method of developing and selecting therapeutic approaches for the use of existing accepted drugs for short term therapy and prevention of long term sequellae of stroke comprising; analysis of samples for subjects post recovery from a stroke and creation of a database according to claim 6; analyzing the database for pathways contributing to the biochemical separation of the group; defining the biochemistry of model systems analyzed according to claim 6; testing of compounds that modulate these pathways in these defined cell, invertebrate and vertebrate models; comparing the pathway effects across the model and human data bases.

33. A method according to claim 1, where the sample is selected from urine, CSF, plasma, nasal swabs, sweat or other body fluid.

34. A method according to claim 2, where the sample is selected from urine, CSF, plasma, nasal swabs, sweat or other body fluid.

35. A method according to claim 3, where the sample is selected from urine, CSF, plasma, nasal swabs, sweat or other body fluid.

36. A method according to claim 4, where the sample is selected from urine, CSF, plasma, nasal swabs, sweat or other body fluid.

37. A method according to claim 5, where the sample is selected from urine, CSF, plasma, nasal swabs, sweat or other body fluid.

38. A method according to claim 6, where the sample is selected from urine, CSF, plasma, nasal swabs, sweat or other body fluid.

39. A method according to claim 7, where the sample is selected from urine, CSF, plasma, nasal swabs, sweat or other body fluid.

40. A method according to claim 31, where the sample is selected from urine, CSF, plasma, nasal swabs, sweat or other body fluid.

41. A method according to claim 32, where the sample is selected from urine, CSF, plasma, nasal swabs, sweat or other body fluid.
